SEQUENCE LISTING (10 sequences)

SEQ ID NO: 1 - DNA, 516 bp, Artificial Sequence
  Description of Artificial Sequence Synthetic polynucleotide
atgcatcctgaaacattggtaaaggtaaaggacgcagaggaccagttaggtgcccgtgtgggatacatcgagttggatcttaattctgggaaaattttagagagcttccgtccggaggaacgattcccgatgatgtcaacttttaaggtacttttgtgcggggccgttttgagccgtatcgacgctggacaggagcagttaggacgacgaatacattactcacagaatgaccttgtggaatacagtcctgttaccgagaaacaccttaccgatgggatgacagtacgtgagttatgctctgctgcaataacgatgtctgacaacacggcagcaaacttattacttacaaccattggagggccaaaggaattaaccgcctttttgcacaatatgggcgatcatgtaaccagattggatagatgggagccggagttgaacgaagcaattccaaacgatgagagagatactacgactcctgtagcaatggcgaccactttgcgaaaacttttgacggga

SEQ ID NO: 2 - protein, 172 aa, Artificial Sequence
  Description of Artificial Sequence Synthetic polypeptide
MHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTG

SEQ ID NO: 3 - DNA, 666 bp, Artificial Sequence
  Description of Artificial Sequence Synthetic polynucleotide
atgaaaaaaaagattattagtgcgatccttatgagtactgttatccttagtgctgccgccccgctttctggggtctatgctgcgcatattgttatggtggacgcctataagcctacgaaaggagggggtggatcaggtggcggcgggtctatgcatcctgaaacattggtaaaggtaaaggacgcagaggaccagttaggtgcccgtgtgggatacatcgagttggatcttaattctgggaaaattttagagagcttccgtccggaggaacgattcccgatgatgtcaacttttaaggtacttttgtgcggggccgttttgagccgtatcgacgctggacaggagcagttaggacgacgaatacattactcacagaatgaccttgtggaatacagtcctgttaccgagaaacaccttaccgatgggatgacagtacgtgagttatgctctgctgcaataacgatgtctgacaacacggcagcaaacttattacttacaaccattggagggccaaaggaattaaccgcctttttgcacaatatgggcgatcatgtaaccagattggatagatgggagccggagttgaacgaagcaattccaaacgatgagagagatactacgactcctgtagcaatggcgaccactttgcgaaaacttttgacggga

SEQ ID NO: 4 - protein, 222 aa, Artificial Sequence
  Description of Artificial Sequence Synthetic polypeptide
MKKKIISAILMSTVILSAAAPLSGVYAAHIVMVDAYKPTKGGGGSGGGGSMHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTG

SEQ ID NO: 5 - DNA, 273 bp, Artificial Sequence
  Description of Artificial Sequence Synthetic polynucleotide
ttacttactttagcatctcgacaacagttaatagactggatggaagccgacaaagtggccggtccgcttttgcgatctgcattgccagcagggtggttcatcgccgataaatcaggcgccggggaaagaggaagccgtggcatcatcgccgccttaggtccagacggtaaaccgagtcgaattgtagtcatatacacgacggggagtcaggcaactatggacgagagaaaccgacagattgccgagatcggggccagtttaataaaacattgg

SEQ ID NO: 6 - protein, 91 aa, Artificial Sequence
  Description of Artificial Sequence Synthetic polypeptide
LLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 7 - DNA, 732 bp, Artificial Sequence
  Description of Artificial Sequence Synthetic polynucleotide
atgaaaaaaaagattattagtgcgatccttatgagtactgttatccttagtgctgccgccccgctttctggggtctatgctggtgcgatggttgataccttgtctggccttagttctgagcaagggcagagtggggatatgacaatagaggaggatagcgccacccacattaagttcagtaaacgtgatgaggatggaaaagaattggcgggagccacgatggaacttagagacagcagtgggaaaacgatttctacgtggatcagcgacggccaggttaaggatttctatctttatcctggaaagtacactttcgttgaaacagcagcgcctgacggatacgaagtcgcgacagcaataacctttacagtaaatgaacagggacaagtgactgtaaatggtaaggcgactaaaggggatgcccatataggagggggtggatcaggtggcggcgggtctttacttactttagcatctcgacaacagttaatagactggatggaagccgacaaagtggccggtccgcttttgcgatctgcattgccagcagggtggttcatcgccgataaatcaggcgccggggaaagaggaagccgtggcatcatcgccgccttaggtccagacggtaaaccgagtcgaattgtagtcatatacacgacggggagtcaggcaactatggacgagagaaaccgacagattgccgagatcggggccagtttaataaaacattgg

SEQ ID NO: 8 - protein, 244 aa, Artificial Sequence
  Description of Artificial Sequence Synthetic polypeptide
MKKKIISAILMSTVILSAAAPLSGVYAGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGGGSGGGGSLLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 9 - DNA, 19 bp, Artificial Sequence
  Description of Artificial Sequence Synthetic primer
gtgccagcmgccgcggtaa

SEQ ID NO: 10 - DNA, 20 bp, Artificial Sequence
  Description of Artificial Sequence Synthetic primer
ggactachvgggtwtctaat

(19) United States
(12) Patent Application Publication
(10) Pub. No.: US 2024/0024383 A1
(43) Pub. Date: Jan. 25, 2024

(54) ENGINEERED PROBIOTIC COMPOSITIONS AND USES THEREOF

(71) Applicants: PRESIDENT AND FELLOWS OF HARVARD COLLEGE, Cambridge, MA (US); MASSACHUSETTS INSTITUTE OF TECHNOLOGY, Cambridge, MA (US)

(72) Inventors: Andres Fernando CUBILLOS, Cambridge, MA (US); James J COLLINS, Cambridge, MA (US)

(73) Assignees: PRESIDENT AND FELLOWS OF HARVARD COLLEGE, Cambridge, MA (US); MASSACHUSETTS INSTITUTE OF TECHNOLOGY, Cambridge, MA (US)

(21) Appl. No.: 18/255,203

(22) PCT Filed: Dec. 1, 2021
(86) PCT No.: PCT/US2021/061414

§ 371 Date: May 31, 2023

Related U.S. Application Data
(60) Provisional application No. 63/119,772, filed on Dec. 1, 2020.

(51) Int. Cl. A61K 35/744; A61K 9/00; A61K 38/50; A61K 45/06; C12N 15/74; A61P 1/00; C12R 1/46

(52) U.S. Cl. CPC A61K 35/744; A61K 9/0053; A61K 38/50; A61K 45/06; C12N 15/746; A61P 1/00; C12Y 305/02006; C12R 2001/46

(57) ABSTRACT

Provided herein are compositions and methods comprising engineered microorganisms and their use for locally degrading an antibiotic in the gastrointestinal tract to prevent or limit death of beneficial flora.

### CROSS-REFERENCE TO RELATED APPLICATIONS

This application is a 35 U.S.C. § 371 National Phase Entry Application of International Application No. PCT/US2021/061414 filed Dec. 1, 2021, which designates the U.S. and claims benefit under 35 U.S.C. § 119(e) to U.S. Provisional Application No. 63/119,772, filed Dec. 1, 2020, the contents of each of which are incorporated herein by reference in their entireties.

### GOVERNMENT SUPPORT

This invention was made with government support under HDTRA1-14-1-0006 awarded by the Department of Defense/Defense Threat Reduction Agency. The Government has certain rights in the invention.

### SEQUENCE LISTING

The instant application contains a Sequence Listing which has been submitted electronically in ASCII format and is hereby incorporated by reference in its entirety. Said ASCII copy, created on Jan. 5, 2022, is named 002806-098940WOPT_SL.txt and is 11,282 bytes in size.

### FIELD OF THE INVENTION

The field of the invention relates to probiotic compositions and uses thereof to promote healthy gut microbiota in a subject being treated with or indicated for treatment with an antibiotic.

### BACKGROUND

Antibiotics are life-saving medicines, yet their use negatively impacts the healthy gut microbiota. Most notably antibiotic use in hospitalized patients represents the highest risk factor for contracting Clostridium difficile.

Clostridium difficile is a major cause of diarrhea in healthcare settings, accounting for 10-20% of antibiotic-associated diarrhea. The mortality for Clostridium difficile infection is estimated at 1-2.5%, contributing to 15,000-30,000 deaths annually in the U.S. (Ananthakrishnan, A. N. “Clostridium difficile infection: epidemiology, risk factors and management,” Nat Rev Gastroenterol Hapatol, 8:17-26 (2011); Parkes, G. C. et al., “The mechanisms and efficacy of probiotics in the prevention of Clostridium difficile-associated diarrhea,” Lancet Infect Dis, 9:237-44 (2009); and O'Keefe, S. J., “Tube feeding, the microbiota, and Clostridium difficile infection,” World J Gastroenterol, 16:139-42 (2010)). Antibiotic-induced perturbation of gut microbiota is widely believed to provide C. difficile an undesirable advantage, allowing it to proliferate and elaborate its toxins in the background of a susceptible flora.

### SUMMARY

The compositions and methods described herein are based, in part, on the generation of an engineered microorganism that is delivered in combination with an antibiotic and which is able to degrade the antibiotic locally in the gut. Thus, this microorganism protects the subject from opportunistic infections, such as C. difficile infections by maintaining a heterogeneous or thriving gut microbiome, while permitting effective delivery of an antibiotic to a non-gut target site.

Accordingly, provided herein in one aspect is a composition comprising a microorganism engineered to degrade an antibiotic in the mammalian gut, wherein the microorganism is also engineered to reduce the likelihood of horizontal transmission of its engineered antibiotic-degrading capacity.

In one embodiment of this aspect and all other aspects described herein, the microorganism's engineered antibiotic degrading capacity comprises expression and secretion of an enzyme activity that degrades the antibiotic.

In another embodiment of this aspect and all other aspects provided herein, the enzyme is encoded in first and second parts, on separate, first and second nucleic acid constructs, wherein neither construct on its own encodes active antibiotic-degrading enzyme, and where both parts of the enzyme are needed to provide antibiotic-degrading activity, thereby reducing the likelihood of horizontal transmission of the engineered antibiotic-degrading activity.

In another embodiment of this aspect and all other aspects provided herein, the microorganism is a bacterium or a yeast.

In another embodiment of this aspect and all other aspects provided herein, the first and second constructs encode the first and second parts of the enzyme as first and second fusion polypeptides, each comprising a respective member of a specific binding pair.

In another embodiment of this aspect and all other aspects provided herein, the first and second fusion polypeptides are secreted by the microorganism into its surrounding environment.

In another embodiment of this aspect and all other aspects provided herein, binding of the first and second fusion polypeptides via the respective members of the specific binding pair promotes the physical interaction of the first and second parts of the enzyme and reconstitution of antibiotic-degrading enzymatic activity.

In another embodiment of this aspect and all other aspects provided herein, the respective members of the specific binding pair promote covalent bonding between the first and second fusion polypeptides.

In another embodiment of this aspect and all other aspects provided herein, the antibiotic-degrading activity comprises β lactamase enzyme activity, Staphylococcus aureus mph(C) gene enzyme activity, S. aureus lincosamide nucleotidyltransferase lnu(A) gene enzyme activity, an Enterococcus faecium lnu(b) gene enzyme activity or an Escherichia coli ereB gene enzyme activity.

In another embodiment of this aspect and all other aspects provided herein, the β lactamase is a TEM1 β lactamase.

In another embodiment of this aspect and all other aspects provided herein, the TEM1 β lactamase is encoded in first and second parts, on separate, first and second nucleic acid constructs, wherein the first nucleic acid construct encodes β lactamase fragment (BLF) 1, comprising SEQ ID NO: 1 or 3, and the second nucleic acid construct encodes BLF 2, comprising SEQ ID NO: 5 or 7.

In another embodiment of this aspect and all other aspects provided herein, the first and second constructs encode the first and second parts of the enzyme as first and second fusion polypeptides, respectively, each comprising a respective member of the SpyTag™/SpyCatcher™ specific binding pair.

In another embodiment of this aspect and all other aspects provided herein, the microorganism is an engineered generally regarded as safe (GRAS) microorganism.

In another embodiment of this aspect and all other aspects provided herein, the microorganism is an engineered lactic acid bacterium.

In another embodiment of this aspect and all other aspects provided herein, the microorganism is an engineered Lactococcus lactis bacterium.

In another embodiment of this aspect and all other aspects provided herein, the composition is in a formulation for oral delivery.

Another aspect provided herein is a viable lyophilized microorganism as described in any of the embodiments recited herein.

In one embodiment of this aspect and all other aspects provided herein, the viable lyophilized microorganism or composition thereof is formulated as a pill, tablet or capsule.

Another aspect described herein relates to a method of treating a bacterial infection, the method comprising administering an antibiotic and a probiotic composition as described herein.

In one embodiment of this aspect and all other aspects provided herein, the antibiotic is delivered parenterally or orally.

In another embodiment of this aspect and all other aspects provided herein, the antibiotic is delivered intravenously.

In another embodiment of this aspect and all other aspects provided herein, the probiotic composition as described herein is administered before the antibiotic is administered.

In another embodiment of this aspect and all other aspects provided herein, the antibiotic is delivered parenterally, and the probiotic composition as described herein is administered before or at the same time the antibiotic is delivered.

In another embodiment of this aspect and all other aspects provided herein, the probiotic composition as described herein is administered orally.

In another embodiment of this aspect and all other aspects provided herein, the probiotic composition as described herein is orally administered before the antibiotic is orally administered.

In another embodiment of this aspect and all other aspects provided herein, the antibiotic is a β-lactam antibiotic, and the microorganism in the probiotic composition as described herein is engineered to express a β-lactamase enzyme.

In another embodiment of this aspect and all other aspects provided herein, the antibiotic is selected from one of the following antibiotic classes: penicillins (e.g., penicillin, penicillin G, penicillin V, oxacillin, nafcillin, dicloxacillin, amoxicillin, ampicillin, ticarcillin, and piperacillin), cephalosporins (e.g., cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, and ceftobiprole), carbapenems (e.g., meropenem, doripenem, and ertapenam), or monobactams (e.g., aztreonam).

In another embodiment of this aspect and all other aspects provided herein, the microorganism in the composition promotes the degradation of the antibiotic in the gut, thereby limiting or preventing antibiotic-induced gut dysbiosis.

Another aspect provided herein relates to a method of preventing or limiting an antibiotic-induced dysbiosis in a subject in need of antibiotic administration, the method comprising administering an antibiotic and a probiotic composition comprising a microorganism engineered to degrade an antibiotic in the mammalian gut, wherein the microorganism is also engineered to reduce the likelihood of horizontal transmission of its engineered antibiotic-degrading capacity, or an embodiment thereof.

In another embodiment of this aspect and all other aspects described herein, the probiotic composition is administered orally.

In another embodiment of this aspect and all other aspects described herein, the antibiotic is administered parenterally or orally.

In another embodiment of this aspect and all other aspects described herein, the probiotic composition as described herein is orally administered before the antibiotic is orally administered.

In another embodiment of this aspect and all other aspects described herein, the bacterial infection is an infection with a bacterium sensitive to a β-lactam antibiotic, and the antibiotic is a β-lactam antibiotic.

In another embodiment of this aspect and all other aspects described herein, the antibiotic is selected from a class of antibiotics including penicillins (e.g., penicillin, penicillin G, penicillin V, oxacillin, nafcillin, dicloxacillin, amoxicillin, ampicillin, ticarcillin, and piperacillin), cephalosporins (e.g., cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, and ceftobiprole), carbapenems (e.g., meropenem, doripenem, and ertapenam), or monobactams (e.g., aztreonam).

Also provided herein, in another aspect, is a method of preventing C. difficile pathology in a subject treated with an antibiotic, the method comprising administering a probiotic composition comprising a microorganism engineered to degrade an antibiotic in the mammalian gut, wherein the microorganism is also engineered to reduce the likelihood of horizontal transmission of its engineered antibiotic-degrading capacity, or an embodiment thereof.

In another embodiment of this aspect and all other aspects described herein, the probiotic composition is administered orally.

In another embodiment of this aspect and all other aspects described herein, the antibiotic is administered parenterally or orally.

In another embodiment of this aspect and all other aspects described herein, the probiotic composition is orally administered before the antibiotic is orally administered.

In another embodiment of this aspect and all other aspects described herein, the subject has a bacterial infection comprising a bacterium sensitive to a β-lactam antibiotic, and the antibiotic is a β-lactam antibiotic.

In another embodiment of this aspect and all other aspects described herein, the antibiotic is selected from penicillin, penicillin G, penicillin V, oxacillin, nafcillin, dicloxacillin, amoxicillin, ampicillin, ticarcillin, piperacillin, cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, ceftobiprole, meropenem, doripenem, ertapenam, and aztreonam.

An additional aspect provided herein relates to a system for limiting or preventing antibiotic-induced dysbiosis, the system comprising: (i) a first nucleic acid construct, encoding a first fusion polypeptide comprising a first part of an antibiotic-degrading enzyme, fused to a first member of a specific binding pair, operably linked to sequence permitting expression of the first fusion polypeptide; (ii) a second nucleic acid construct, encoding a second fusion polypeptide comprising a second part of an antibiotic-degrading enzyme, fused to the second member of the specific binding pair, operably linked to sequence permitting expression of the second fusion polypeptide, wherein neither the first fusion polypeptide nor the second fusion polypeptide alone can degrade antibiotic, but wherein a physical association between the first and second fusion polypeptides permits association between the first and second parts of the antibiotic-degrading enzyme to form an active antibiotic-degrading complex.

In one embodiment of this aspect and all other aspects described herein, the system is comprised by a microorganism.

In another embodiment of this aspect and all other aspects described herein, the microorganism is a bacterium or a yeast.

In another embodiment of this aspect and all other aspects described herein, the respective members of the specific binding pair promote covalent bonding between the first and second fusion polypeptides.

In another embodiment of this aspect and all other aspects described herein, the antibiotic-degrading activity comprises a β lactamase enzyme activity, Staphylococcus aureus mph(C) gene enzyme activity, S. aureus lincosamide nucleotidyltransferase lnu(A) gene enzyme activity, an Enterococcus faecium lnu(b) gene enzyme activity or an Escherichia coli ereB gene enzyme activity.

In another embodiment of this aspect and all other aspects described herein, the μlactamase is a TEM1 β lactamase.

In another embodiment of this aspect and all other aspects described herein, the first nucleic acid construct comprises sequence encoding β lactamase fragment (BLF) 1, comprising SEQ ID NO: 1 or 3, and the second nucleic acid construct comprises sequence encoding BLF 2, comprising SEQ ID NO: 5 or 7.

In another embodiment of this aspect and all other aspects described herein, the specific binding pair is the respective members of the SpyTag™/SpyCatcher™ specific binding pair.

In another embodiment of this aspect and all other aspects described herein, the microorganism is an engineered GRAS microorganism.

In another embodiment of this aspect and all other aspects described herein, the microorganism is a lactic acid bacterium.

In another embodiment of this aspect and all other aspects described herein, the microorganism is a Lactococcus lactis bacterium.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B Split β-lactamase (spTEM1) system for extracellular degradation of β-lactam antibiotics. FIG. 1A. Engineered biosynthesis pathway of the spTEM1 system in L. lactis. The ST-BLF-1 and the SC-BLF-2 subunits are expressed from independent genetic loci and actively secreted for extracellular assembly of the active enzyme. FIG. 1B. Nitrocefin hydrolysis assay for the detection of β-lactamase activity. Covalent bond formation between the SpyTag™ (ST) and SpyCatcher™ (SC) domains enhances the rate of assembly of the BLF-1 and BLF-2 subunits and promotes the restoration of enzymatic activity of the spTEM1.

FIGS. 2A-2B. Expression of spTEM1 does not confer ampicillin resistance to single cells. FIG. 2A. Cell density-dependent growth on ampicillin. β-lactamases in the periplasm of Gram-negative bacteria i.e. E. coli, confer selective advantage to single-cells. Survival to ampicillin is an emergent property of the of the L. lactis spTEM1 population and not of single cells. Diffusion from the cell surface precludes protection to the producer cell when the population density is low. FIG. 2B. Estimation of the ampicillin survival density threshold in prototypical Gram-negative and Gram-positive strains expressing elements of the spTEM1 system. Expression of the spTEM1 system in E. coli does not confer resistance to single cells despite the availability of a periplasm.

FIGS. 3A-3E. L. lactis spTEM1 protects the diversity and composition of gut the microbiota in an ampicillin-induced dysbiosis murine model. FIG. 3A. Experimental design to test the efficacy of L. lactis spTEM1 in preventing dysbiosis by parenteral ampicillin in mice. Treatment consisted of one daily dose of 200 mg/kg of ampicillin and two doses of 10¹⁰ CFU of probiotic (2 hours prior and simultaneous with the ampicillin injection) for 3 days. C. difficile infection was performed with 5×10³ spores at 24 hours post-treatment. Fecal samples were collected throughout the experiment to monitor pathogen colonization and microbiota compositions. Blood sample was collected 30 minutes after the last ampicillin dose to measure its concentration in serum. FIG. 3B. Determination of the Shannon diversity index for gut microbial communities in mice pre- and post-treatment. The p-values correspond to unpaired t-test between the groups that received L. lactis spTEM1 and L. lactis EV. FIG. 3C. Principal Coordinates Analysis of the beta-diversity between gut microbial communities in mice pre- and post-treatment. FIG. 3D. Supplementation of the mouse gut with L. lactis in the absence of ampicillin does not alter the diversity of the gut microbiota. FIG. 3E. Analysis of Composition of Microbiomes (ANCOM) identifies differential abundance of bacterial populations pre- and post-ampicillin in mice treated with L. lactis spTEM1 or L. lactis EV. Dashed line indicates the significance cutoff value for the ANCOM W value. Differential increase or decrease is reported with respect to the first condition stated. Resistant or permissive indicates the status of the C. difficile colonization resistance observed for that group. n=8 mice in each L. lactis spTEM1 and L. lactis EV groups. n=5 mice for ampicillin-naive mice supplemented with L. lactis EV. ns, not significant paired t-test.

FIGS. 4A-4B. L. lactis spTEM1 prevents the enrichment of ARG following the administration of ampicillin in mice. FIG. 4A. Analysis of the abundance of ARG reveals significant enrichment in ampicillin-treated mice receiving L. lactis EV but not in mice receiving L. lactis spTEM1. Stacked bar data is presented as reads mapping the different CARD database categories and is normalized to the size of the read pool in each sample. Vector-derived ARG in the β-lactam and chloramphenicol classes are presented as a different category to differentiate them from endogenous ARG. The p-values correspond to paired t-test between pre-treatment and the later time points. *** is p<0.0001. FIG. 4B. Abundance of endogenous and probiotic-derived β-lactamases in the mouse. Elimination of the ampicillin selective pressure by the spTEM1 β-lactamases reduces the enrichment of endogenous β-lactamases in the mouse gut. Rapid elimination from the system of the spTEM1 genes comparted to endogenous β-lactamase genes suggests lack of competitive advantage in the spTEM1 strain. Statistical significance was calculated with a negative binomial generalized linear model with Tukey's post hoc test between pre-treatment and later time points for each probiotic strain. The adjusted p-values are ** p<0.01, *** p<0.001. n=8 mice in each L. lactis spTEM1 and L. lactis EV groups.

FIGS. 5A-5D. L. lactis spTEM1 maintains the colonization resistance against C. difficile in ampicillin-treated mice. FIG. 5A. Engineered probiotic intervention aimed at protecting the native gut microbiota as a preventative for the loss of colonization resistance against C. difficile. FIG. 5B. Enumeration of viable C. difficile cells in mouse feces at 24 hours and 48 hours post-infection. Data correspond to two independent experiments for ampicillin-treated mice in the spTEM1 and EV groups, N=12; experiment 1 n=4 and experiment 2 n=8. For ampicillin-free mice in the EV group N=10; experiment 1 n=5 and experiment 2 n=5. FIG. 5C. Quantification of ampicillin in serum after 30 minutes of the last dose reveals no significant differences between mice dosed with the β-lactamase expressing probiotic and the control. FIG. 5D. Enumeration of viable L. lactis cells in feces 4 hours after the first dose in each day of treatment demonstrates no difference in the fecal loads of L. lactis spTEM1 (n=8) and L. lactis EV (n=8).

FIG. 6. Detection of 3-lactamase activity in components of the spTEM1 system using a nitrocefin hydrolysis assay. Nanobodies, recombinant antigen-binding proteins derived from single-chain camelid antibodies, were also used to promote reconstitution of the beta-lactamase fragments. The LaM-4 nanobody (see e.g., Fridy et al. Nature Methods (2014) 11:1253-1260) was fused to the BLF1 fragment and its cognate antigen, the mCherry protein, was fused to the BLF2 fragment. Interaction between the LaM4 nanobody and the mCherry protein enhances the rate of assembly of the BLF1 and BLF2 subunits and promotes the restoration of enzymatic activity of the spTEM1.

FIGS. 7A-7C. Mouse model for parenteral ampicillin-induced dysbiosis and the disruption of colonization resistance against C. difficile. FIG. 7A. A 3-day intraperitoneal ampicillin administration regimen is evaluated for its effects in abolishing colonization resistance against 5×10³ spores of C. difficile at 24 hours after the last ampicillin dose. C. difficile density in feces is evaluated 24 hours after the infection. FIG. 7B. Evaluation of single or double dose (8 hours apart) administration regimens for intraperitoneal ampicillin injection indicates that a single dose of ampicillin for 3 days is enough to sensitize the mouse gut to robust C. difficile colonization. FIG. 7C. Dose-dependency of single daily intraperitoneal ampicillin injections in the disruption of colonization resistance against C. difficile.

FIG. 8. Rarefaction plot for all samples indicate that species diversity was near saturation at the chosen sampling depth (59900).

### DETAILED DESCRIPTION

Provided herein are compositions and methods comprising an engineered microorganism that can protect the gut microbiota of a subject from an antibiotic by degrading that antibiotic. The engineered microorganism does not affect the action of the antibiotic at other sites in the subject. In addition, the engineered microorganism is generated such that horizontal gene transfer of this antibiotic degrading enzyme to a native microorganism is prevented, thus reducing or eliminating the potential for generation of new antibiotic-resistant species in the host.

### Definitions

The terms “patient,” “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.). It is specifically contemplated herein that a subject can be of any developmental age including, but not limited to, a fetus, a neonate, an infant, a toddler, a child, an adolescent, an adult, post-menopausal, or a geriatric subject.

As used herein, the term “subject in need of antibiotic administration” refers to a subject having a bacterial infection and/or one for whom antibiotic administration is indicated, e.g., for prophylaxis prior to or coincident with a dental, surgical or other therapeutically indicated procedure.

As used herein, the term “prophylactic treatment” refers to administration of the engineered microorganisms as described herein in a subject that is indicated for (e.g., at risk of a bacterial infection) or being administered a given antibiotic, but does not yet have an active bacterial infection.

As used herein, the terms “administering,” and “introducing” are used interchangeably in the context of the placement of cells, e.g., a composition comprising an engineered microorganism as described herein into a subject, by a method or route which results in at least partial localization of the introduced cells at a desired site, such as the intestines or a region thereof, such that a desired effect(s) is produced (e.g., local degradation of an antibiotic in the gastrointestinal tract). The cells can be administered by any appropriate route which results in delivery to a desired location in the subject where at least a portion of the delivered cells or components of the cells remain viable. The period of viability of the cells after administration to a subject can be as short as a few hours, e.g., six to twenty-four hours, to a few days, to as long as several years, i.e., long-term engraftment. In one embodiment, the engineered microorganism is transient and does not engraft or colonize the gastrointestinal tract for a substantial amount of time. Thus, a transient engineered microorganism may require multiple administrations to maintain the antibiotic-degrading action over the length of time necessary for the course of antibiotics to be complete and/or for the “wash-out” period of the antibiotic from the gastrointestinal tract of the subject being treated.

As used herein “preventing” or “prevention” refers to any methodology where the disease state does not occur due to the actions of the methodology (such as, for example, administration of a composition as described herein). In one aspect, it is understood that prevention can also mean that the bacterial infection is not established to the extent that occurs in untreated controls. For example, there can be a 5, 10, 15, 20, 25, 30, 35, 40, 50, 60, 70, 80, 90, or 100% reduction in the establishment of bacterial infection frequency relative to untreated controls. Accordingly, prevention of a bacterial infection (e.g., C. difficile) encompasses a reduction in the likelihood that a subject will develop the bacterial infection, relative to an untreated subject (e.g. a subject who is not treated with a composition as described herein).

As used herein, the term “antibiotic degrading” refers to any modification that inactivates the cytotoxic/anti-microbial effect of the antibiotic, for example, by catalyzing the cleavage of a covalent bond, or addition of a moiety that interferes with the cytotoxic/antimicrobial effect of the antibiotic molecule; an “antibiotic degrading” enzyme does not necessarily have to break the antibiotic down into component parts.

As used herein, the phrase “effective amount” means an amount sufficient to achieve a meaningful benefit (e.g., reducing antibiotic concentration in the gut; reduced risk and/or incidence of C. difficile infection; maintenance of commensal bacteria in the gastrointestinal tract).

As used herein, “stably maintained” or “stable” bacterium is used to refer to a bacterial host cell carrying non-native genetic material, (e.g., gene cassettes expressing, separately, each of the subunits of the antibiotic degrading enzyme) that is incorporated into the host genome or propagated on a self-replicating extra-chromosomal plasmid or plasmids, such that the non-native genetic material is retained, expressed, and propagated. The stable bacterium is capable of survival and/or growth in vitro, e.g., in medium, and/or in vivo, e.g., in the gut.

The term “probiotic” is used herein to refer to live, non-pathogenic microorganisms, e.g., bacteria or yeast, which can confer health benefits to a subject that contains an appropriate amount of the microorganism. In some embodiments, the subject is a mammal. In some embodiments, the subject is a human. Typically, a probiotic microorganism will be classified as ‘generally recognized as safe” (GRAS) according to the U.S. Federal Drug Administration (FDA), or will meet the requirements for such classification. Some species, strains, and/or subtypes of non-pathogenic bacteria are currently recognized as probiotic. Examples of probiotic bacteria include, but are not limited to non-pathogenic species of, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). Any of these non-pathogenic bacteria can be used to generate the engineered microorganisms described herein.

The terms “decrease”, “reduced”, “reduction”, or “inhibit” are all used herein to mean a decrease or lessening of a property, level, or other parameter (such as a biological marker or a disease symptom) by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient, allows the ingredient to retain biological activity and is substantially non-reactive with the subject's immune system (unless desired). Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture and bacterial culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

### Microorganisms

Essentially any non-pathogenic bacterium or yeast (or those modified to be non-pathogenic) can be used to generate engineered microorganisms as described herein. Thus, provided herein are methods and compositions that comprise non-pathogenic bacteria engineered to express an antibiotic-degrading enzyme. Such non-pathogenic bacteria include, but are not limited to, probiotic bacteria or yeast.

Some species, strains, and/or subtypes of non-pathogenic bacteria or yeast are currently recognized as probiotics. Examples of probiotics include, but are not limited to certain non-pathogenic Candida spp., Debaryomyces spp., Debaryomyces spp., Enterococcus spp., Kluyveromyces spp., Kluyveromyces spp., Saccharomyces spp., Yarrowia spp., Bifidobacteria spp., Escherichia coli, Vagococcus spp., Carnobacterium spp., Melissococcus spp. and Lactobacillus spp. Exemplary probiotic strains include e.g., Candida humilis, Debaryomyces hansenii, Debaryomyces occidentalis, Kluyveromyces lactis, Kluyveromyces lodderae, Kluyveromyces marxianus, Saccharomyces cerevisiae, Saccharomyces boulardii, Yarrowia hpolytica, Bifidobacterium bifidum, Enterococcus faecium, Enterococcus faecalis, Enterococcus hirae, Enterococcus casseliflavus, Enterococcus gallinarum, Escherichia coli strain Nissle, Lactobacillus acidophilus, Lactobacillus bulgaricus, Lactobacillus paracasei, Lactobacillus plantarum, Vagococcus fluvaialis (Dinleyici et al., 2014; U.S. Pat. Nos. 5,589,168; 6,203,797; 6,835,376). The probiotic can be a variant or a mutant strain of bacterium (Arthur et al., 2012; Cuevas-Ramos et al., 2010; Olier et al., 2012; Nougayrede et al., 2006).

In another embodiment, the engineered bacteria or yeast are engineered from a species classified as “generally recognized as safe” (GRAS) by the United States Food and Drug Administration (FDA).

In another embodiment, the engineered microorganisms described herein are engineered from a species having a “qualified presumption of safety” (QPS) status as defined by the European Food Safety Authority (EFSA). An introduction of the qualified presumption of safety (QPS) approach for the assessment of selected microorganisms is described in the EFSA Journal, Vol. 587, 2007, pages 1-16.

In one embodiment, the engineered microorganisms described herein comprise bacteria. In another embodiment, the engineered microorganisms described herein consist essentially of bacteria. In certain embodiments, the non-pathogenic bacteria belong to the phylum firmicutes or actinobacteria. Other exemplary non-pathogenic bacteria include those from at least one genus selected from the group consisting of Bifidobacterium, Corynebacterium, Enterococcus, Lactobacillus, Lactococcus, Leuconostoc, Pediococcus, Propionibacterium, and Streptococcus.

In other embodiments, the engineered microorganisms or compositions thereof comprise lactic acid bacteria (LAB). Lactic acid bacteria are a Clade of gram-positive, acid-tolerant, generally non-sporulating, non-respiring bacteria that share common metabolic and physiological characteristics. These bacteria produce lactic acid as a major metabolic end product of carbohydrate metabolism. Furthermore, lactic acid bacteria have a generally recognized as safe (GRAS) status due to their ubiquitous appearance in food and their contribution to the healthy microflora of human mucosal surfaces.

In another embodiment, the probiotic bacteria are from the genus Bifidobacterium sp., including but not limited to, Bifidobacterium actinocoloniiforme, Bifidobacterium adolescentis, Bifidobacterium aesculapii, Bifidobacterium angulatum, Bifidobacterium animalis, for example Bifidobacterium animalis subsp. animalis or Bifidobacterium animalis subsp. lactis, Bifidobacterium asteroides, Bifidobacterium biavatii, Bifidobacterium bifidum, Bifidobacterium bohemicum, Bifidobacterium bombi, Bifidobacterium boum, Bifidobacterium breve, Bifidobacterium callitrichos, Bifidobacterium catenulatum, Bifidobacterium choerinum, Bifidobacterium coryneforme, Bifidobacterium crudilactis, Bifidobacterium cuniculi, Bifidobacterium denticolens, Bifidobacterium dentium, Bifidobacterium faecale, Bifidobacterium gallicum, Bifidobacterium gallinarum, Bifidobacterium globosum, Bifidobacterium indicum, Bifidobacterium infantis, Bifidobacterium inopinatum, Bifidobacterium kashiwanohense, Bifidobacterium lactis, Bifidobacterium longum, for example Bifidobacterium longum subsp. infantis, Bifidobacterium longum subsp. longum, or Bifidobacterium longum subsp. suis, Bifidobacterium magnum, Bifidobacterium merycicum, Bifidobacterium minimum, Bifidobacterium mongoliense, Bifidobacterium moukalabense, Bifidobacterium pseudocatenulatum, Bifidobacterium pseudolongum, for example Bifidobacterium pseudolongum subsp. globosum or Bifidobacterium pseudolongum subsp. pseudolongum, Bifidobacterium psychraerophilum, Bifidobacterium pullorum, Bifidobacterium reuteri, Bifidobacterium ruminantium, Bifidobacterium saeculare, Bifidobacterium saguini, Bifidobacterium scardovii, Bifidobacterium stellenboschense, Bifidobacterium subtile, Bifidobacterium stercoris, Bifidobacterium suis, Bifidobacterium thermacidophilum, for example Bifidobacterium thermacidophilum subsp. porcinum or Bifidobacterium thermacidophilum subsp. thermacidophilum, Bifidobacterium thermophilum, or Bifidobacterium tsurumiense.

In some embodiments, the probiotic bacteria are bacteria having a “Qualified Presumption of Safety” (QPS) status in the genus Bifidobacterium sp., including but not limited to, Bifidobacterium adolescentis, Bijfdobacterium animalis, Bifidobacterium longum, Bifidobacterium breve, or Bifidobacterium bifidum.

In other embodiments, engineered microorganisms described herein are from the genus Corynebacterium sp., including but not limited to, Corynebacterium accolens, Corynebacterium afermentans, for example Corynebacterium afermentans subsp. afermentans or Corynebacterium afermentans subsp. lipophilum, Corynebacterium ammoniagenes, Corynebacterium amycolatum, Corynebacterium appendices, Corynebacterium aquatimens, Corynebacterium aquilae, Corynebacterium argentoratense, Corynebacterium atypicum, Corynebacterium aurimucosum, Corynebacterium auris, Corynebacterium auriscanis, Corynebacterium betae, Corynebacterium beticola, Corynebacterium bovis, Corynebacterium callunae, Corynebacterium camporealensis, Corynebacterium canis, Corynebacterium capitovis, Corynebacterium casei, Corynebacterium caspium, Corynebacterium ciconiae, Corynebacterium confusum, Corynebacterium coyleae, Corynebacterium cystitidis, Corynebacterium deserti, Corynebacterium diphtheriae, Corynebacterium doosanense, Corynebacterium durum, Corynebacterium efficiens, Corynebacterium epidermidicanis, Corynebacterium equi, Corynebacterium falsenii, Corynebacterium fascians, Corynebacterium felinum, Corynebacterium faccumfaciens, for example Corynebacterium flaccumfaciens subsp. betae, Corynebacterium flaccumfaciens subsp. flaccumfaciens, Corynebacterium flaccumfaciens subsp. oortii, or Corynebacterium faccumfaciens subsp. poinsettiae, Corynebacterium flavescens, Corynebacterium frankenforstense, Corynebacterium freiburgense, Corynebacterium freneyi, Corynebacterium glaucum, Corynebacterium glucuronolyticum, Corynebacterium glutamicum, Corynebacterium halotolerans, Corynebacterium hansenii, Corynebacterium hoagie, Corynebacterium humireducens, Corynebacterium ilicis, Corynebacterium imitans, Corynebacterium insidiosum, Corynebacterium iranicum, Corynebacterium jeikeium, Corynebacterium kroppenstedtii, Corynebacterium kutscheri, Corynebacterium lactis, Corynebacterium lilium, Corynebacterium lipophiloflavum, Corynebacterium liquefaciens, Corynebacterium lubricantis, Corynebacterium macginleyi, Corynebacterium marinum, Corynebacterium marls, Corynebacterium massiliense, Corynebacterium mastitidis, Corynebacterium matruchotii, Corynebacterium michiganense, for example Corynebacterium michiganense subsp. insidiosum, Corynebacterium michiganense subsp. michiganense, Corynebacterium michiganense subsp. nebraskense, Corynebacterium michiganense subsp. sepedonicum, or Corynebacterium michiganense subsp. tessellarius, Corynebacterium minutissimum, Corynebacterium mooreparkense, Corynebacterium mucifaciens, Corynebacterium mustelae, Corynebacterium mycetoides, Corynebacterium nebraskense, Corynebacterium nigricans, Corynebacterium nuruki, Corynebacterium oortii, Corynebacterium paurometabolum, Corynebacterium phocae, Corynebacterium pilbarense, Corynebacterium pilosum, Corynebacterium poinsettiae, Corynebacterium propinquum, Corynebacterium pseudodiphtheriticum, Corynebacterium pseudotuberculosis, Corynebacterium pyogenes, Corynebacterium pyruviciproducens, Corynebacterium rathayi, Corynebacterium renale, Corynebacterium resistens, Corynebacterium riegelii, Corynebacterium seminale, Corynebacterium sepedonicum, Corynebacterium simulans, Corynebacterium singulare, Corynebacterium sphenisci, Corynebacterium spheniscorum, Corynebacterium sputi, Corynebacterium stationis, Corynebacterium striatum, Corynebacterium suicordis, Corynebacterium sundsvallense, Corynebacterium terpenotabidum, Corynebacterium testudinoris, Corynebacterium thomssenii, Corynebacterium timonense, Corynebacterium tritici, Corynebacterium tuberculostearicum, Corynebacterium tuscaniense, Corynebacterium ulcerans, Corynebacterium ulceribovis, Corynebacterium urealyticum, Corynebacterium ureicelerivorans, Corynebacterium uterequi, Corynebacterium variabile, Corynebacterium vitaeruminis, or Corynebacterium xerosis.

In another embodiment, the engineered microorganisms are generated from bacteria from the genus Enterococcus sp., including but not limited to, Enterococcus alcedinis, Enterococcus aquimarinus, Enterococcus asini, Enterococcus avium, Enterococcus caccae, Enterococcus camelliae, Enterococcus canintestini, Enterococcus canis, Enterococcus casseliflavus, Enterococcus cecorum, Enterococcus columbae, Enterococcus devriesei, Enterococcus diestrammenae, Enterococcus dispar, Enterococcus durans, Enterococcus eurekensis, Enterococcus faecalis, Enterococcus faecium, Enterococcus flavescens, Enterococcus gallinarum, Enterococcus gilvus, Enterococcus haemoperoxidus, Enterococcus hermanniensis, Enterococcus hirae, Enterococcus italicus, Enterococcus lactis, Enterococcus lemanii, Enterococcus malodoratus, Enterococcus moraviensis, Enterococcus mundtii, Enterococcus olivae, Enterococcus pallens, Enterococcus phoeniculicola, Enterococcus plantarum, Enterococcus porcinus, Enterococcus pseudoavium, Enterococcus quebecensis, Enterococcus raffinosus, Enterococcus ratti, Enterococcus rivorum, Enterococcus rotai, Enterococcus saccharolyticus, for example Enterococcus saccharolyticus subsp. saccharolyticus or Enterococcus saccharolyticus subsp. taiwanensis, Enterococcus saccharominimus, Enterococcus seriolicida, Enterococcus silesiacus, Enterococcus solitarius, Enterococcus sulfureus, Enterococcus termitis, Enterococcus thailandicus, Enterococcus ureilyticus, Enterococcus viikkiensis, Enterococcus villorum, or Enterococcus xiangfangensis.

In some embodiments, the engineered bacteria are generated from bacteria classified as “generally regarded as safe” (GRAS) in the Enterococcus genus, including but not limited to Enterococcus durans, Enterococcus faecalis, or Enterococcus faecium.

In other embodiments, the engineered microorganisms described herein are generated from bacteria of the genus Lactobacillus sp., including but not limited to, Lactobacillus acetotolerans, Lactobacillus acidifarinae, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylotrophicus, Lactobacillus amylovorus, Lactobacillus animalis, Lactobacillus antri, Lactobacillus apinorum, Lactobacillus apis, Lactobacillus apodemi, Lactobacillus aquaticus, Lactobacillus arizonensis, Lactobacillus aviaries, for example Lactobacillus aviarius subsp. araffinosus or Lactobacillus aviarius subsp. aviarius, Lactobacillus backii, Lactobacillus bavaricus, Lactobacillus bifermentans, Lactobacillus bobalius, Lactobacillus bombi, Lactobacillus brantae, Lactobacillus brevis, Lactobacillus buchneri, Lactobacillus bulgaricus, Lactobacillus cacaonum, Lactobacillus camelliae, Lactobacillus capillatus, Lactobacillus carnis, Lactobacillus casei, for example Lactobacillus casei subsp. alactosus, Lactobacillus casei subsp. casei, Lactobacillus casei subsp. pseudoplantarum, Lactobacillus casei subsp. rhamnosus, or Lactobacillus casei subsp. tolerans, Lactobacillus catenaformis, Lactobacillus cellobiosus, Lactobacillus ceti, Lactobacillus coleohominis, Lactobacillus collinoides, Lactobacillus composti, Lactobacillus concavus, Lactobacillus confusus, Lactobacillus coryniformis, for example Lactobacillus coryniformis subsp. coryniformis or Lactobacillus coryniformis subsp. torquens, Lactobacillus crispatus, Lactobacillus crustorum, Lactobacillus curieae, Lactobacillus curvatus, for example Lactobacillus curvatus subsp. curvatus or Lactobacillus curvatus subsp. melibiosus, Lactobacillus cypricasei, Lactobacillus delbrueckii, for example Lactobacillus delbrueckii subsp. bulgaricus, Lactobacillus delbrueckii subsp. delbrueckii, Lactobacillus delbrueckii subsp. indicus, Lactobacillus delbrueckii subsp. jakobsenii, Lactobacillus delbrueckii subsp. lactis, or Lactobacillus delbrueckii subsp. sunkii, Lactobacillus dextrinicus, Lactobacillus diolivorans, Lactobacillus divergens, Lactobacillus durianis, Lactobacillus equi, Lactobacillus equicursoris, Lactobacillus equigenerosi, Lactobacillus fabifermentans, Lactobacillus faecis, Lactobacillus farciminis, Lactobacillus farraginis, Lactobacillus ferintoshensis, Lactobacillus fermentum, Lactobacillus floricola, Lactobacillus forum, Lactobacillus fornicalis, Lactobacillus fructivorans, Lactobacillus fructosus, Lactobacillus frumenti, Lactobacillus fuchuensis, Lactobacillus furfuricola, Lactobacillus futsaii, Lactobacillus gallinarum, Lactobacillus gasseri, Lactobacillus gastricus, Lactobacillus ghanensis, Lactobacillus gigeriorum, Lactobacillus graminis, Lactobacillus halotolerans, Lactobacillus hammesii, Lactobacillus hamsteri, Lactobacillus harbinensis, Lactobacillus hayakitensis, Lactobacillus heilongjiangensis, Lactobacillus helsingborgensis, Lactobacillus helveticus, Lactobacillus heterohiochii, Lactobacillus hilgardii, Lactobacillus hokkaidonensis, Lactobacillus hominis, Lactobacillus homohiochii, Lactobacillus hordei, Lactobacillus iners, Lactobacillus ingluviei, Lactobacillus intestinalis, Lactobacillus iwatensis, Lactobacillus jensenii, Lactobacillus johnsonii, Lactobacillus kalixensis, Lactobacillus kandleri, Lactobacillus kefiranofaciens, for example Lactobacillus kefiranofaciens subsp. kefiranofaciens or Lactobacillus kefiranofaciens subsp. kefirgranum, Lactobacillus kefiri, Lactobacillus kefirgranum, Lactobacillus kimbladii, Lactobacillus kimchicus, Lactobacillus kimchiensis, Lactobacillus kimchii, Lactobacillus kisonensis, Lactobacillus kitasatonis, Lactobacillus koreensis, Lactobacillus kullabergensis, Lactobacillus kunkeei, Lactobacillus lactis, Lactobacillus leichmannii, Lactobacillus lindneri, Lactobacillus malefermentans, Lactobacillus mali, Lactobacillus maltaromicus, Lactobacillus manihotivorans, Lactobacillus mellfer, Lactobacillus mellis, Lactobacillus melliventris, Lactobacillus mindensis, Lactobacillus minor, Lactobacillus minutus, Lactobacillus mucosae, Lactobacillus murinus, Lactobacillus nagelii, Lactobacillus namurensis, Lactobacillus nantensis, Lactobacillus nasuensis, Lactobacillus nenjiangensis, Lactobacillus nodensis, Lactobacillus odoratitofui, Lactobacillus oeni, Lactobacillus oligofermentans, Lactobacillus oris, Lactobacillus oryzae, Lactobacillus otakiensis, Lactobacillus ozensis, Lactobacilluspanis, Lactobacillus pantheris, Lactobacillus parabrevis, Lactobacillus parabuchneri, Lactobacillus paracasei, for example Lactobacillus paracasei subsp. paracasei or Lactobacillus paracasei subsp. tolerans, Lactobacillus paracollinoides, Lactobacillus parafarraginis, Lactobacillus parakefiri, Lactobacillus paralimentarius, Lactobacillus paraplantarum, Lactobacillus pasteurii, Lactobacillus paucivorans, Lactobacillus pentosus, Lactobacillus perolens, Lactobacillus piscicola, Lactobacillus plantarum, for example Lactobacillus plantarum subsp. argentoratensis or Lactobacillus plantarum subsp. plantarum, Lactobacillus pobuzihii, Lactobacillus pontis, Lactobacillus porcinae, Lactobacillus psittaci, Lactobacillus rapi, Lactobacillus rennini, Lactobacillus reuteri, Lactobacillus rhamnosus, Lactobacillus rimae, Lactobacillus rodentium, Lactobacillus rogosae, Lactobacillus rossiae, Lactobacillus ruminis, Lactobacillus saerimneri, Lactobacillus sakei, for example Lactobacillus sakei subsp. carnosus or Lactobacillus sakei subsp. sakei, Lactobacillus salivarius, for example Lactobacillus salivarius subsp. salicinius or Lactobacillus salivarius subsp. salivarius, Lactobacillus sanfranciscensis, Lactobacillus saniviri, Lactobacillus satsumensis, Lactobacillus secaliphilus, Lactobacillus selangorensis, Lactobacillus senioris, Lactobacillus senmaizukei, Lactobacillus sharpeae, Lactobacillus shenzhenensis, Lactobacillus sicerae, Lactobacillus silagei, Lactobacillus siliginis, Lactobacillus similes, Lactobacillus sobrius, Lactobacillus songhuajiangensis, Lactobacillus spicheri, Lactobacillus sucicola, Lactobacillus suebicus, Lactobacillus sunkii, Lactobacillus suntoryeus, Lactobacillus taiwanensis, Lactobacillus thailandensis, Lactobacillus thermotolerans, Lactobacillus trichodes, Lactobacillus tucceti, Lactobacillus uli, Lactobacillus ultunensis, Lactobacillus uvarum, Lactobacillus vaccinostercus, Lactobacillus vaginalis, Lactobacillus versmoldensis, Lactobacillus vini, Lactobacillus viridescens, Lactobacillus vitulinus, Lactobacillus xiangfangensis, Lactobacillus xylosus, Lactobacillus yamanashiensis, for example Lactobacillus yamanashiensis subsp. mali or Lactobacillus yamanashiensis subsp. yamanashiensis, Lactobacillus yonginensis, Lactobacillus zeae, or Lactobacillus zymae.

In certain embodiments, the engineered bacteria are bacteria classified as “generally regarded as safe” (GRAS) in the genus Lactobacillus sp., including but not limited to, Lactobacillus acidophilus strain NP 28, Lactobacillus acidophilus strain NP51, Lactobacillus subsp. lactis strain NP7, Lactobacillus reuteri strain NCIMB 30242, Lactobacillus casei strain Shirota, Lactobacillus reuteri strain DSM17938, Lactobacillus reuteri strain NCIMB 30242, Lactobacillus acidophilus strain NCFM, Lactobacillus rhamnosus strain HN001, Lactobacillus rhamnosus strain HN001, Lactobacillus reuteri strain DSM 17938, Lactobacillus casei subsp. rhamnosus strain GG, Lactobacillus acidophilus, Lactobacillus lactis, Lactobacillus acetotolerans, Lactobacillus acidifarinae, Lactobacillus acidipiscis, Lactobacillus acidophilus, Lactobacillus alimentarius, Lactobacillus amylolyticus, Lactobacillus amylovorus, Lactobacillus brevis, Lactobacillus buchneri, Lactobacillus cacaonum, Lactobacillus casei subsp. casei, Lactobacillus collinoides, Lactobacillus composti, Lactobacillus coryniformis subsp. coryniformis, Lactobacillus crispatus, Lactobacillus crustorum, Lactobacillus curvatus subps. curvatus, Lactobacillus delbrueckii subsp. bulgaricus, Lactobacillus delbrueckii subsp. delbrueckii, Lactobacillus delbrueckii subsp. lactis, Lactobacillus dextrinicus, Lactobacillus diolivorans, Lactobacillus fabifermentans, Lactobacillus farciminis, Lactobacillus fermentum, Lactobacillus fructivorans, Lactobacillus frumenti, Lactobacillus gasseri, Lactobacillus ghanensis, Lactobacillus hammesii, Lactobacillus harbinensis, Lactobacillus helveticus, Lactobacillus hilgardii, Lactobacillus homohiochii, Lactobacillus hordei, Lactobacillus jensenii, Lactobacillus johnsonii, Lactobacillus kefiri, Lactobacillus kefiranofadens subsp. kefiranofaciens, Lactobacillus kefiranofadens subsp. kefirgranum, Lactobacillus kimchii, Lactobacillus kisonensis, Lactobacillus mail, Lactobacillus manihotivorans, Lactobacillus mindensis, Lactobacillus mucosae, Lactobacillus nagelii, Lactobacillus namurensis, Lactobacillus nantensis, Lactobacillus nodensis, Lactobacillus oeni, Lactobacillus otakiensis, Lactobacillus panis, Lactobacillus parabrevis, Lactobacillus parabuchneri, Lactobacillus paracasei subsp. paracasei, Lactobacillus parakefiri, Lactobacillus paralimentarius, Lactobacillus paraplantarum, Lactobacillus pentosus, Lactobacillus perolens, Lactobacillus plantarum subsp. plantarum, Lactobacillus pobuzihii, Lactobacillus pontis, Lactobacillus rapi, Lactobacillus reuteri, Lactobacillus rhamnosus, Lactobacillus rossiae, Lactobacillus sakei subsp carnosus, Lactobacillus sakei subsp. sakei, Lactobacillus sali varius subsp. salivarius, Lactobacillus sanfranciscensis, Lactobacillus satsumensis, Lactobacillus secaliphilus, Lactobacillus senmaizukei, Lactobacillus siliginis, Lactobacillus spicheri, Lactobacillus suebicus, Lactobacillus sunkii, Lactobacillus tucceti, Lactobacillus vaccinostercus, Lactobacillus versmoldensis, or Lactobacillus yamanashiensis.

In some embodiments, the engineered bacteria are bacteria having a “Qualified Presumption of Safety” (QPS) status in the genus Lactobacillus sp., including but not limited to, Lactobacillus acidophilus, Lactobacillus amylolyticus, Lactobacillus amylovorus, Lactobacillus alimentarius, Lactobacillus aviaries, Lactobacillus brevis, Lactobacillus buchneri, Lactobacillus casei, Lactobacillus crispatus, Lactobacillus curvatus, Lactobacillus delbrueckii, Lactobacillus farciminis, Lactobacillus fermentum, Lactobacillus gallinarum, Lactobacillus gasseri, Lactobacillus helveticus, Lactobacillus hilgardii, Lactobacillus johnsonii, Lactobacillus kefiranofaciens, Lactobacillus kefiri, Lactobacillus mucosae, Lactobacillus panis, Lactobacillus paracasei, Lactobacillus paraplantarum, Lactobacillus pentosus, Lactobacillus plantarum, Lactobacillus pontis, Lactobacillus reuteri, Lactobacillus rhamnosus, Lactobacillus sakei, Lactobacillus salivarius, Lactobacillus sanfranciscensis, or Lactobacillus zeae.

In other embodiments, the engineered microorganisms are from the genus Lactococcus sp., including but not limited to Lactococcus chungangensis, Lactococcus formosensis, Lactococcus fujiensis, Lactococcus garvieae, Lactococcus lactis, for example Lactococcus lactis subsp. cremoris, Lactococcus lactis subsp. hordniae, Lactococcus lactis subsp. lactis, or Lactococcus lactis subsp. tructae, Lactococcus piscium, Lactococcus plantarum, Lactococcus raffrnolactis, or Lactococcus taiwanensis.

In other embodiments, the engineered bacteria are bacteria classified as “generally regarded as safe” (GRAS) in the genus Lactococcus, including but not limited to, Lactococcus lactis subsp. cremoris, Lactococcus lactis subsp. lactis, and Lactococcus raffinolactis.

In some embodiments, the engineered bacteria are Lactococcus lactis, Lactococcus lactis subsp. lactis, Lactococcus lactis subsp. lactis biovariant diacetylactis, or Lactococcus lactis subsp. cremoris, or Lactococcus lactis subsp. cremoris.

In another embodiment, the engineered bacteria are bacteria from the genus Leuconostoc sp., including but not limited to, Leuconostoc amelibiosum, Leuconostoc argentinum, Leuconostoc carnosum, Leuconostoc citreum, Leuconostoc cremoris, Leuconostoc dextranicum, Leuconostoc durionis, Leuconostoc fallax, Leuconostoc ficulneum, Leuconostoc fructosum, Leuconostoc gasicomitatum, Leuconostoc gelidum, for example Leuconostoc gelidum subsp. aenigmaticum, Leuconostoc gelidum subsp. gasicomitatum, or Leuconostoc gelidum subsp. gelidum, Leuconostoc holzapfelii, Leuconostoc inhae, Leuconostoc kimchii, Leuconostoc lactis, Leuconostoc mesenteroides, for example Leuconostoc mesenteroides subsp. cremoris, Leuconostoc mesenteroides subsp. dextranicums, Leuconostoc mesenteroides subsp. mesenteroides, or Leuconostoc mesenteroides subsp. suionicum, Leuconostoc miyukkimchii, Leuconostoc oeni, Leuconostoc paramesenteroides, Leuconostoc pseudoficulneum, or Leuconostoc pseudomesenteroides.

Preferably, the bacteria are bacteria classified as “generally regarded as safe” (GRAS) in the genus Leuconostoc sp., including but not limited to, Leuconostoc carnosum, Leuconostoc citreum, Leuconostoc fallax, Leuconostoc holzapfelii, Leuconostoc inhae, Leuconostoc kimchii, Leuconostoc lactis, Leuconostoc mesenteroides subsp. cremoris, Leuconostoc mesenteroides subsp. dextranicums, Leuconostoc mesenteroides subsp. mesenteroides, Leuconostoc palmae, or Leuconostoc pseudomesenteroides. Alternatively, the engineered bacteria are bacteria having a “Qualified Presumption of Safety” (QPS) status in the genus Leuconostoc sp., including but not limited to, Leuconostoc citreum, Leuconostoc lactis, Leuconostoc mesenteroides subsp. cremoris, Leuconostoc mesenteroides subsp. dextranicum, or Leuconostoc mesenteroides subsp. mesenteroides.

In another embodiment, the bacteria are from the genus of Pediococcus sp., including but not limited to, Pediococcus acidilactici, Pediococcus argentinicus, Pediococcus cellicola, Pediococcus claussenii, Pediococcus damnosus, Pediococcus dextrinicus. Pediococcus ethanolidurans, Pediococcus halophilus, Pediococcus inopinatus, Pediococcus lohi, Pediococcus parvulus, Pediococcus pentosaceus, Pediococcus siamensis, Pediococcus stilesii, or Pediococcus urinaeequi.

In some embodiments, the engineered bacteria are generated from bacteria having a “Qualified Presumption of Safety” (QPS) status in the genus Pediococcus sp., including but not limited to, Pediococcus acidilactici, Pediococcus dextrinicus, or Pediococcus pentosaceus.

In another embodiment, the bacteria from which the engineered bacteria are derived are from the genus Propionibacterium sp., including but not limited to, Propionibacterium acidifaciens, Propionibacterium acidipropionici, Propionibacterium acnes, Propionibacterium australiense, Propionibacterium avidum, Propionibacterium cyclohexanicum, Propionibacterium damnosum, Propionibacterium freudenreichii, for example Propionibacterium freudenreichii subsp. freudenreichii or Propionibacterium freudenreichii subsp. shermanii, Propionibacterium granulosum, Propionibacterium innocuum, Propionibacterium jensenii, Propionibacterium lymphophilum, Propionibacterium microaerophilum, Propionibacterium olivae, Propionibacterium propionicum, or Propionibacterium thoenii.

In other embodiments, the bacteria used to generate an engineered bacterium are bacteria classified as “generally regarded as safe” (GRAS) in the genus Propionibacterium sp., including but not limited to Propionibacterium acidipropionici, Propionibacterium freudenreichii subsp. Freudenreichii, Propionibacterium freudenreichii subsp. shermanii, Propionibacterium jensenii, or Propionibacterium thoenii.

In some embodiments, bacteria used to generate the engineered microorganisms described herein are Propionibacterium freudenreichii, Propionibacterium freudenreichii subsp. freudenreichii or Propionibacterium freudenreichii subsp. shermanii.

In other embodiments, the engineered microorganisms described herein are generated from bacteria from the genus Streptococcus sp., including but not limited to, Streptococcus acidominimus, Streptococcus adjacens, Streptococcus agalactiae, Streptococcus alactolyticus, Streptococcus anginosus, Streptococcus australis, Streptococcus bovis, Streptococcus caballi, Streptococcus canis, Streptococcus caprinus, Streptococcus castoreus, Streptococcus cecorum, Streptococcus constellatus, for example Streptococcus constellatus subsp. constellatus, Streptococcus constellatus subsp. pharynges, or Streptococcus constellatus subsp. viborgensis, Streptococcus cremoris, Streptococcus criceti, Streptococcus cristatus, Streptococcus cuniculi, Streptococcus danieliae, Streptococcus defectivus, Streptococcus dentapri, Streptococcus dentirousetti, Streptococcus dentasini, Streptococcus dentisani, Streptococcus devriesei, Streptococcus didelphis, Streptococcus difficilis, Streptococcus downei, Streptococcus durans, Streptococcus dysgalactiae, for example Streptococcus dysgalactiae subsp. dysgalactiae or Streptococcus dysgalactiae subsp. equisimilis, Streptococcus entericus, Streptococcus equi, for example Streptococcus equi subsp. equi, Streptococcus equi subsp. ruminatorum, or Streptococcus equi subsp. zooepidemicus, Streptococcus equinus, Streptococcus faecalis, Streptococcus faecium, Streptococcus ferus, Streptococcus gallinaceus, Streptococcus gallinarum, Streptococcus gallolyticus, for example Streptococcus gallolyticus subsp. gallolyticus, Streptococcus gallolyticus subsp. macedonicus, or Streptococcus gallolyticus subsp. pasteurianus, Streptococcus garvieae, Streptococcus gordonii, Streptococcus halichoeri, Streptococcus hansenii, Streptococcus henryi, Streptococcus hongkongensis, Streptococcus hyointestinalis, Streptococcus hyovaginalis, Streptococcus ictaluri, Streptococcus infantarius, for example Streptococcus infantarius subsp. coli or Streptococcus infantarius subsp. infantarius, Streptococcus infantis, Streptococcus iniae, Streptococcus intermedius, Streptococcus intestinalis, Streptococcus lactarius, Streptococcus lactis, for example Streptococcus lactis subsp. cremoris, Streptococcus lactis subsp. diacetilactis, or Streptococcus lactis subsp. lactis, Streptococcus loxodontisalivarius, Streptococcus lutetiensis, Streptococcus macacae, Streptococcus macedonicus, Streptococcus marimammalium, Streptococcus massiliensis, Streptococcus merionis, Streptococcus minor, Streptococcus mitis, Streptococcus morbillorum, Streptococcus moroccensis, Streptococcus mutans, Streptococcus oligofermentans, Streptococcus oralis, Streptococcus orisasini, Streptococcus orisuis, Streptococcus ovis, Streptococcus parasanguinis, Streptococcus parauberis, Streptococcus parvulus, Streptococcus pasteurianus, Streptococcus peroris, Streptococcus phocae, for example Streptococcus phocae subsp. phocae or Streptococcus phocae subsp. salmonis, Streptococcus plantarum, Streptococcus pleomorphus, Streptococcus pluranimalium, Streptococcus plurextorum, Streptococcus pneumonia, Streptococcus porci, Streptococcus porcinus, Streptococcus porcorum, Streptococcus pseudopneumoniae, Streptococcus pseudoporcinus, Streptococcus pyogenes, Streptococcus raffinolactis, Streptococcus ratti, Streptococcus rifensis, Streptococcus rubneri, Streptococcus rupicaprae, Streptococcus saccharolyticus, Streptococcus salivarius, for example Streptococcus salivarius subsp. salivarius or Streptococcus salivarius subsp. thermophilus, Streptococcus saliviloxodontae, Streptococcus sanguinis, Streptococcus shiloi, Streptococcus sinensis, Streptococcus sobrinus, Streptococcus suis, Streptococcus thermophilus, Streptococcus thoraltensis, Streptococcus tigurinus, Streptococcus troglodytae, Streptococcus uberis, Streptococcus urinalis, Streptococcus ursoris, Streptococcus vestibularis, or Streptococcus waius.

In certain embodiments, engineered bacteria are a bacteria classified as “generally regarded as safe” (GRAS) in the genus Streptococcus sp., including but not limited to, Streptococcus thermophilus strain Th4, Streptococcus gallolyticus subsp. macedonicus, Streptococcus salivarius subsp. salivarius, or Streptococrus salivarius subsp. thermophilus.

In some embodiments, the engineered bacteria are bacteria having a “Qualified Presumption of Safety” (QPS) status in the genus Streptococcus sp., including but not limited to, Streptococcus thermophilus.

As will be recognized by one of skill in the art, preferred bacteria are those that do not produce endotoxins or other potentially toxic substances. Similarly, bacteria that do not produce spores are preferred so that the bacteria cannot survive, for example, without nutrients or if an auxotrophic factor is missing. In some embodiments, the engineered bacteria do not produce inclusion bodies that could contain over-expressed proteins causing aggregation of the proteins, which can limit the utility of the bacteria.

In some embodiments, the engineered bacteria do not produce extracellular proteinases. Extracellular proteinases can be secreted from bacteria to destroy extracellular structures, such as proteins, to generate nutrients, such as carbon, nitrogen, or sulfur. Extracellular proteinases may also act as an exotoxin and be an example of a virulence factor in bacterial pathogenesis.

The engineered microorganisms described herein are modified such that antibiotic resistance cannot be transferred to other commensal or environmental bacteria. Thus, in some embodiments, the engineered bacteria lack host factors required for conjugative transposition (i.e., sexual conjugation between different cells).

In some embodiments, the engineered microorganisms described herein comprise yeast. Essentially any non-pathogenic or probiotic yeast can be used to generate yeast-based engineered microorganisms as described herein. Exemplary yeast strains include, but are not limited to, Saccharomyces cerevisiae, Saccharomyces boulardii, Saccharomyces pastorianus, Saccharomyces bayanus, Kluveromyces marxianus and the like.

### Preparing Engineered Microorganisms

Provided herein are engineered microorganisms and compositions thereof for reducing the risk or incidence of dysbiosis including, but not limited to e.g., C. difficile infection, in a subject being treated with an antibiotic. In one embodiment, the engineered microorganism is an engineered yeast cell or population thereof. In another embodiment, the engineered microorganism is an engineered bacterial cell or population thereof.

The microorganisms that are described herein are engineered to express an antibiotic-degrading enzyme. The antibiotic-degrading enzyme is expressed from at least two different expression cassettes such that the individually expressed components come together to from the antibiotic-degrading enzyme. Expression of the enzyme components from at least two different expression cassettes prevents single gene horizontal gene transfer to other commensal bacteria or environmental bacteria, as well as limiting any selective advantage to the engineered microorganism in the gastrointestinal tract.

In some embodiments, the engineered microorganisms are naturally non-pathogenic bacteria. However, in some embodiments, the engineered microorganisms can be naturally pathogenic bacteria that are modified or mutated to reduce or eliminate pathogenicity. In some embodiments, the engineered microorganisms are commensal bacteria. In some embodiments, the engineered microorganisms are probiotic bacteria.

Engineered microorganisms can also harbor one or more introduced genetic changes, such change being an insertion, deletion, translocation, or substitution, or any combination thereof, of one or more nucleotides contained on the bacterial chromosome or on an endogenous plasmid, wherein the genetic change can result in the alteration, disruption, removal, or addition of one or more protein coding genes, non-protein-coding genes, gene regulatory regions, or any combination thereof, and wherein such change can be a fusion of two or more separate genomic regions or can be synthetically derived.

The engineered microorganisms can be produced using techniques including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, or any combination thereof.

In order to facilitate transfer of genetic material (e.g., a plasmid, DNA etc.) to a bacterium, artificial cell competence can be induced by exposing the bacterium to particular conditions. For example, one method of inducing cell competence is by incubating the bacterium in a solution with divalent cations (e.g., calcium chloride) to partially disrupt the membrane and then heat-shocking the host cells to induce them to take up e.g., plasmid DNA. An alternative method for inducing cell competence is electroporation, where the cells are exposed to an electric field, which can generate small holes in the cell membrane such that plasmid DNA can enter the cell.

The plasmid-supplied nucleic acid (e.g., DNA) can be stably integrated into the genome or can be maintained episomally, e.g., on a plasmid or other episomal vector. In some embodiments, a sequence directing the expression of the one or more enzymes described herein can be placed under the control of naturally-occurring regulatory elements in the cell. In other embodiments, constructs for the expression of the components of the antibiotic-degrading enzyme will generally include regulatory elements, including promoters, enhancers, etc. that direct the expression of the encoded sequences. A gene under the control of a set of regulatory elements is generally referred to as “operably linked” to those elements. Typically, an expression vector comprises a transcription promoter, a transgene encoding sequence, and a transcription terminator. In some embodiments, one or both of the at least two expression cassettes are operably linked to a directly or indirectly inducible promoter (i.e., the same or different promoters). In some embodiments, the inducible promoter(s) is not associated with the gene or gene cassette in nature.

An expression cassette or expression vector as described herein is a nucleic acid molecule encoding a component of an antibiotic-degrading enzyme that is expressed when the molecule is introduced to a host-cell.

In some embodiments it can be useful to include in the transformed cells a positive marker that permits the selection of cells positive for a plasmid-supplied nucleic acid sequence(s) (i.e., the expression cassette or expression vector) in vitro. The positive selectable marker can be a gene that upon being introduced into the host cell expresses a dominant phenotype permitting positive selection of cells carrying the gene. Genes of this type are known in the art, and include, for example, an antibiotic resistance gene (e.g., resistance to blasticidin, mycophenolic acid, puromycin, zeocin, actinomycin, ampicillin, kanamycin, neomycin, polymixin B sulfate, or streptomycin), or an enzyme that converts a substrate to a colored product (e.g., blue/white screening by expression of B-galactosidase, which converts X-gal into galactose and an insoluble blue pigment) etc. Other selection tools can include e.g., radioactive nucleic acid probes, and labeled antibodies that are specific to the protein expressed by the transgene.

### Antibiotic-Degrading Enzymes

The engineered microorganisms as described herein are engineered to express an antibiotic-degrading enzyme. As will be appreciated by those of skill in the art, such engineered microorganisms require safety measures to prevent the development of antibiotic resistance by the engineered microorganism itself or other commensal or environmental microorganisms. Thus, in one embodiment, the antibiotic-degrading enzyme is expressed as at least two different fragments that are re-constituted intracellularly or extracellularly to generate an active antibiotic-degrading enzyme. The separation of the enzyme into different fragments expressed from genetic elements that are not closely linked in the genome aids in eliminating the potential for horizontal gene transfer as a single genetic element.

This approach can be applied generally to sequence encoding antibiotic-degrading enzymes. That is, the approach is applicable to an antibiotic-degrading enzyme that can be expressed in parts, from separate genetic elements that permit the encoded polypeptides to associate to reconstitute the active enzyme activity. Homology modeling using esterases involved in succinoglycan biosynthesis can be used to determine appropriate separation regions of a given antibiotic-degrading enzyme (i.e., a homologous antibiotic-degrading enzyme). For example, such homology modeling of macrolide esterases revealed conserved catalytic residues (H40, E68, and H279) implicated in the hydrolysis process (see e.g., Golkar et al. Front Microbiol (2018) 9:1942. doi: 10.3389/fmicb.2018.01942). The construction of a split biosynthesis strategy for macrolide esterases is possible when preserving the structural configuration of these predicted catalytic residues. As another example, analysis of the secondary structure in the Escherichia coli ereB polypeptide reveals the presence of multiple potential split points where the main alpha-helices and beta-sheets are not likely to be perturbed and can be used as breakpoints to fuse the dimerization domains that will enable a split biosynthesis strategy.

The structural properties and key catalytic residues of a wide-variety of β-lactamases have been extensively studied (see e.g., Palskill Front Mol Biosci (2018) 23(5):16). Class A μ-lactamases, which are serine active-site hydrolases that include, but are not limited to, TEM, CTX-M, and KPC enzymes, show a remarkably high degree of structural conservation. Individual class A β-lactamases display a range of substrate specificities that altogether encompasses almost all clinically used penicillins, cephalosporins, and carbapenems (see e.g., Palskill, supra). Given the highly conserved secondary structures with the class A β-lactamases, the split biosynthesis approach developed for the TEM1 enzyme and described in the working examples herein can be readily extrapolated to other members of the class A β-lactamase group. In particular, the residue at position 297 can be used as the breakpoint for the generation of the analogous BLF1 and BLF2 polypeptides in other Class A 3-lactamase enzymes.

In some embodiments, a targeted mutation approach is used. Rather than relying upon the identification of new degrading enzymes to express as split proteins, one can modify existing and well-known enzymes to recognize and cleave different substrate antibiotics of the same class. For example, a comprehensive review of the mutations observed for the TEM1 β-lactamase in clinical isolates and in experimental evolution studies, and their resulting effects on β-lactam substrate specificity serves as a guide to identify residues that can be modified to alter the range of the β-lactams that can be inactivated with this enzyme (see e.g., Salverda et al. FEMS Microbiol Rev (2010) 34(6):1015-1036). This information serves as a base for targeted mutagenesis approaches to modify the spectrum of μ-lactams that the spTEM1 system can target. For instance, mutation of the following residues (according to the Ambler notation) has functional consequences for the substrate specificity of the TEM1 enzyme: M69, E104, S130, R164, M182, A237, G238, E240, R244 and N276.

Exemplary antibiotic-degrading enzyme activities include, but are not limited to, β-lactamase enzyme activity, Staphylococcus aureus mph(C) gene enzyme activity, lincosamide-modifying enzymes (see, e.g., Petinaki et al., Antimicrob. Agents Chemother. 52: 626-630 (2008) and Brisson-Noel et al., J. Biol. Chem. 263: 15880-15887 (1988) and Morar et al. Structure 17: 1649-1659 (2009), exemplified by S. aureus lincosamide nucleotidyltransferase lnu(A) and lnu(E) gene and Enterococcus faecium lnu(b) gene enzyme activities) and ereB gene enzyme activity, e.g., E. coli ereB gene enzyme activity. The S. aureus mph(C) gene enzyme is active against erythromycin, the lnu(A)-(E) gene enzymes are active against particular lincosamides, and the ereB gene encodes an esterase that confers resistance to erythromycin through inactivation by hydrolyzing the lactone ring of the antibiotic. Sequences and/or x-ray crystal structures for these and other antibiotic-degrading enzymes are known and available in the art. The design of split enzymes for each of these can include analysis of the secondary structures, e.g., alpha helix, beta sheet, etc. and separation of the structures at a junction of such structural elements. This separation, combined with fusion to members of a specific binding pair as described herein or as known in the art, can provide a split enzyme system in which the fused members of the specific binding pair bring the respective portions of the antibiotic-degrading enzyme into proximity to permit their interaction to reconstitute the functional antibiotic-degrading enzyme.

In one embodiment, the enzyme activity comprises β-lactamase activity. In one embodiment, the β-lactamase enzyme comprises TEM1 β lactamase. As exemplified herein, the TEM1 β lactamase can be encoded in first and second parts, on separate, first and second nucleic acid constructs, wherein the first nucleic acid construct encodes β lactamase fragment (BLF) 1, comprising SEQ ID NO: 1, and the second nucleic acid construct encodes BLF 2, comprising SEQ ID NO: 5. The BLF1 and BLF2-encoding sequences exemplified by SEQ ID NO: 3 and SEQ ID NO: 7 each further comprise a secretion signal to direct the expressed product out of the cell.

>BLF1 nt
(SEQ ID NO: 1)
ATGCATCCTGAAACATTGGTAAAGGTAAAGGACGCAGAGGACCAGTTAGG
TGCCCGTGTGGGATACATCGAGTTGGATCTTAATTCTGGGAAAATTTTAG
AGAGCTTCCGTCCGGAGGAACGATTCCCGATGATGTCAACTTTTAAGGTA
CTTTTGTGCGGGGCCGTTTTGAGCCGTATCGACGCTGGACAGGAGCAGTT
AGGACGACGAATACATTACTCACAGAATGACCTTGTGGAATACAGTCCTG
TTACCGAGAAACACCTTACCGATGGGATGACAGTACGTGAGTTATGCTCT
GCTGCAATAACGATGTCTGACAACACGGCAGCAAACTTATTACTTACAAC
CATTGGAGGGCCAAAGGAATTAACCGCCTTTTTGCACAATATGGGCGATC
ATGTAACCAGATTGGATAGATGGGAGCCGGAGTTGAACGAAGCAATTCCA
AACGATGAGAGAGATACTACGACTCCTGTAGCAATGGCGACCACTTTGCG
AAAACTTTTGACGGGA
>BLF1 aa
(SEQ ID NO: 2)
MHPETLVKVKDAEDQLGARVGYIELDLNSGKILESFRPEERFPMMSTFKV
LLCGAVLSRIDAGQEQLGRRIHYSQNDLVEYSPVTEKHLTDGMTVRELCS
AAITMSDNTAANLLLTTIGGPKELTAFLHNMGDHVTRLDRWEPELNEAIP
NDERDTTTPVAMATTLRKLLTG
>ST-BLF1 nt
(SEQ ID NO: 3)
ATGAAAAAAAAGATTATTAGTGCGATCCTTATGAGTACTGTTATCCTTAG
TGCTGCCGCCCCGCTTTCTGGGGTCTATGCTGCGCATATTGTTATGGTGG
ACGCCTATAAGCCTACGAAAGGAGGGGGTGGATCAGGTGGCGGCGGGTCT
ATGCATCCTGAAACATTGGTAAAGGTAAAGGACGCAGAGGACCAGTTAGG
TGCCCGTGTGGGATACATCGAGTTGGATCTTAATTCTGGGAAAATTTTAG
AGAGCTTCCGTCCGGAGGAACGATTCCCGATGATGTCAACTTTTAAGGTA
CTTTTGTGCGGGGCCGTTTTGAGCCGTATCGACGCTGGACAGGAGCAGTT
AGGACGACGAATACATTACTCACAGAATGACCTTGTGGAATACAGTCCTG
TTACCGAGAAACACCTTACCGATGGGATGACAGTACGTGAGTTATGCTCT
GCTGCAATAACGATGTCTGACAACACGGCAGCAAACTTATTACTTACAAC
CATTGGAGGGCCAAAGGAATTAACCGCCTTTTTGCACAATATGGGCGATC
ATGTAACCAGATTGGATAGATGGGAGCCGGAGTTGAACGAAGCAATTCCA
AACGATGAGAGAGATACTACGACTCCTGTAGCAATGGCGACCACTTTGCG
AAAACTTTTGACGGGA
>ST-BLF1 aa
(SEQ ID NO: 4)
MKKKIISAILMSTVILSAAAPLSGVYAAHIVMVDAYKPTKGGGGSGGGGS
MHPETLVKVKDAEDQLGARVGYIELDLNSGKILESFRPEERFPMMSTFKV
LLCGAVLSRIDAGQEQLGRRIHYSQNDLVEYSPVTEKHLTDGMTVRELCS
AAITMSDNTAANLLLTTIGGPKELTAFLHNMGDHVTRLDRWEPELNEAIP
NDERDTTTPVAMATTLRKLLTG
>BLF2 nt
(SEQ ID NO: 5)
TTACTTACTTTAGCATCTCGACAACAGTTAATAGACTGGATGGAAGCCGA
CAAAGTGGCCGGTCCGCTTTTGCGATCTGCATTGCCAGCAGGGTGGTTCA
TCGCCGATAAATCAGGCGCCGGGGAAAGAGGAAGCCGTGGCATCATCGCC
GCCTTAGGTCCAGACGGTAAACCGAGTCGAATTGTAGTCATATACACGAC
GGGGAGTCAGGCAACTATGGACGAGAGAAACCGACAGATTGCCGAGATCG
GGGCCAGTTTAATAAAACATTGG
>BLF2 aa
(SEQ ID NO: 6)
LLTLASRQQLIDWMEADKVAGPLLRSALPAGWFIADKSGAGERGSRGIIA
ALGPDGKPSRIVVIYTTGSQATMDERNRQIAEIGASLIKHW
>SC-BLF2 nt
(SEQ ID NO: 7)
ATGAAAAAAAAGATTATTAGTGCGATCCTTATGAGTACTGTTATCCTTAG
TGCTGCCGCCCCGCTTTCTGGGGTCTATGCTGGTGCGATGGTTGATACCT
TGTCTGGCCTTAGTTCTGAGCAAGGGCAGAGTGGGGATATGACAATAGAG
GAGGATAGCGCCACCCACATTAAGTTCAGTAAACGTGATGAGGATGGAAA
AGAATTGGCGGGAGCCACGATGGAACTTAGAGACAGCAGTGGGAAAACGA
TTTCTACGTGGATCAGCGACGGCCAGGTTAAGGATTTCTATCTTTATCCT
GGAAAGTACACTTTCGTTGAAACAGCAGCGCCTGACGGATACGAAGTCGC
GACAGCAATAACCTTTACAGTAAATGAACAGGGACAAGTGACTGTAAATG
GTAAGGCGACTAAAGGGGATGCCCATATAGGAGGGGGTGGATCAGGTGGC
GGCGGGTCTTTACTTACTTTAGCATCTCGACAACAGTTAATAGACTGGAT
GGAAGCCGACAAAGTGGCCGGTCCGCTTTTGCGATCTGCATTGCCAGCAG
GGTGGTTCATCGCCGATAAATCAGGCGCCGGGGAAAGAGGAAGCCGTGGC
ATCATCGCCGCCTTAGGTCCAGACGGTAAACCGAGTCGAATTGTAGTCAT
ATACACGACGGGGAGTCAGGCAACTATGGACGAGAGAAACCGACAGATTG
CCGAGATCGGGGCCAGTTTAATAAAACATTGG
>SC-BLF2 aa
(SEQ ID NO: 8)
MKKKIISAILMSTVILSAAAPLSGVYAGAMVDTLSGLSSEQGQSGDMTIE
EDSATHIKFSKRDEDGKELAGATMELRDSSGKTISTWISDGQVKDFYLYP
GKYTFVETAAPDGYEVATAITFTVNEQGQVTVNGKATKGDAHIGGGGSGG
GGSLLTLASRQQLIDWMEADKVAGPLLRSALPAGWFIADKSGAGERGSRG
IIAALGPDGKPSRIVVIYTTGSQATMDERNRQIAEIGASLIKHW

In certain embodiments, the β lactamase activity permits degradation and/or inactivation of at least one antibiotic in the gut, wherein the at least one antibiotic is from a class of antibiotics that comprise a βlactam ring, for example, penicillins, cephalosporins, carbapenems, monobactams, or β-lactamase inhibitors.

In one embodiment, the at least one penicillin antibiotic is selected from the group consisting of: penicillin, penicillin G, penicillin V, oxacillin, nafcillin, dicloxacillin, amoxicillin, ampicillin, ticarcillin, and piperacillin.

In one embodiment, the at least one cephalosporin antibiotic is selected from the group consisting of: cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, and ceftobiprole.

In one embodiment, the at least one carbapenem antibiotic is selected from the group consisting of: meropenem, doripenem, and ertapenam.

In one embodiment, the at least one monobactam antibiotic is aztreonam.

In one embodiment, the engineered microorganisms express a β lactamase the permits degradation and/or inactivation of one or more antibiotics of an administered combination of antibiotics. Exemplary antibiotic combinations include, but are not limited to, ceftazidime/avibactam, ceftolozone/tazobactam, imipenem/cilastatin, ceftolozane/tazobactam, ceftazidime/avibactam, meropenem/vaborbactam, imipenem/cilastatin/relebactam, or aztreonam/avibactam.

In order to permit reconstitution of an active antibiotic-degrading enzyme following expression of at least two fragments of the enzyme, each fragment can be attached to a member of a specific binding pair (e.g., dimerization domains). Specific protein binding pairs can include, but are not limited to, nanobody/antigen targets (e.g., LaM2 Nanobody and the BC2 Nanobody), leucine zipper domains, SpyTag™/SpyCatcher™, SnoopTag™/SnoopCatcher™, or split intein pairs. In some embodiments, the dimerization domains do not induce covalent bond formation. While this approach facilitates reconstitution of enzyme activity by bringing the separated portions of the enzyme into proximity, the separate enzyme portions associate and dissociate at a rate that depends on the nature of the specific binding pair domain interaction, and the efficiency of enzyme reconstitution and antibiotic degradation depends in large part upon that rate. In another embodiment, a covalent bond-forming dimerization domain can be used to permit reconstitution of the antibiotic-degrading enzyme. The SpyTag™/SpyCatcher™, SnoopTag™/SnoopCatcher™ approach to stimulating covalent joining between the separated enzyme portions, or analogous covalent joining approaches, can overcome the on/off rate issue seen with non-covalent rejoining approaches. When a yeast strain is used to generate the engineered microorganisms, an scFv/antigen pair can be used, however this is not optimal for expression in bacteria.

In certain embodiments, the specific protein binding pair comprises a split intein pair. An “intein” is an intervening protein domain that excises itself from a host protein in a traceless manner during a post-translational autoprocessing event (see e.g., Stevens, A J J Am Chem Soc (2016) 138(7):2162-2165). The flanking polypeptide sequences (exteins) are ligated together via a normal peptide bond following the removal of the intein. Certain inteins exist naturally in a split form. The two pieces of the split intein are separately expressed and remain inactive until encountering their complementary partner, upon which they cooperatively fold and undergo splicing in trans.

In certain embodiments, rather than joining the separated portions of the enzyme at the protein level, the separately encoded portions of the enzyme can be joined at the transcript level, with RNA encoding the active antibiotic-degrading enzyme reconstituted such that the antibiotic-degrading enzyme is expressed as one unit. This embodiment can be achieved using, for example, bacterial group II introns to splice the two fragment transcripts (see e.g., Belhocine, et al. RNA (2008) 14(9):1782-1790; Belhocine et al. Nucleic Acids Res (2007) 35(7):2257-2268). Having the two encoding genes far apart in the DNA will help to prevent horizontal gene transmission.

### Secretion

In some embodiments, the engineered microorganisms are engineered bacteria. In some such embodiments, the engineered bacteria described herein further comprise a non-native secretion mechanism that is capable of secreting the expressed antibiotic-degrading enzyme from the bacterial cytoplasm. In some embodiments, the bacteria comprise a system to transport the antibiotic-degrading enzyme out of the periplasm. It is specifically contemplated herein that engineered bacteria as described herein secrete the antibiotic-degrading enzyme or its component parts outside of the periplasm such that it can leave the immediate vicinity of the cell that secreted it. Secretion beyond the periplasmic space permits the antibiotic-degrading action of the enzyme and prevents the engineered microorganism from having a growth advantage over other commensal microorganisms. In one embodiment, the enzyme is not anchored to the cell wall. In some embodiments, the enzyme is reconstituted intracellularly and then secreted out of the cell. In other embodiments, the enzyme is secreted outside of the cell where it is then extracellularly reconstituted.

Many bacteria have evolved sophisticated secretion systems to transport substrates across the bacterial cell envelope.

In Gram-negative bacteria, secretion machineries can span one or both of the inner and outer membranes. In some embodiments, the engineered bacteria further comprise a non-native double membrane-spanning secretion system. Double membrane-spanning secretion systems include, but are not limited to, the type I secretion system (T1SS), the type II secretion system (T2SS), the type III secretion system (T3SS), the type IV secretion system (T4SS), the type VI secretion system (T6SS), and the resistance-nodulation-division (RND) family of multi-drug efflux pumps (Pugsley 1993; Gerlach et al., 2007; Collinson et al., 2015; Costa et al., 2015; Reeves et al., 2015; WO2014138324A1, incorporated herein by reference in its entirety). Mycobacteria, which have a Gram-negative-like cell envelope, may also encode a type VII secretion system (T7SS) (Stanley et al., 2003). With the exception of the T2SS, double membrane-spanning secretions generally transport substrates from the bacterial cytoplasm directly into the extracellular space or into the target cell. In contrast, the T2SS and secretion systems that span only the outer membrane may use a two-step mechanism, wherein substrates are first translocated to the periplasm by inner membrane-spanning transporters, and then transferred to the outer membrane or secreted into the extracellular space. Outer membrane-spanning secretion systems include, but are not limited to, the type V secretion or autotransporter system (T5SS), the curli secretion system, and the chaperone-usher pathway for pili assembly (Saier, 2006; Costa et al., 2015).

In some embodiments, the engineered bacteria as described herein further comprise a type III or a type Ill-like secretion system (T3SS) from Shigella, Salmonella, E. coli, Vibrio, Burkholderia, Yersinia, Chlamydia, or Pseudomonas. The T3SS is capable of transporting aprotein from the bacterial cytoplasm to the host cytoplasm through a needle complex. The T3SS can be modified to secrete the molecule from the bacterial cytoplasm, but not inject the molecule into the host cytoplasm. In some embodiments, the engineered bacteria comprise such a modified T3SS and are capable of secreting the antibiotic-degrading enzyme or parts thereof from the bacterial cytoplasm.

In alternative embodiments, the engineered bacteria can comprise a non-native single membrane-spanning secretion system. Single membrane-spanning transporters can act as a component of a secretion system, or can export substrates independently. Such transporters include, but are not limited to, ATP-binding cassette translocases, flagellum/virulence-related translocases, conjugation-related translocases, the general secretory system (e.g., the SecYEG complex in E. coli), the accessory secretory system in mycobacteria and several types of Gram-positive bacteria (e.g., Bacillus anthracis, Lactobacillus johnsonii, Corynebacterium glutamicum, Streptococcus gordonii, Staphylococcus aureus), and the twin-arginine translocation (TAT) system (Saier, 2006; Rigel and Braunstein, 2008; Albiniak et al., 2013). It is known that the general secretory and TAT systems can both export substrates with cleavable N-terminal signal peptides into the periplasm, and have been explored in the context of biopharmaceutical production. The TAT system may offer particular advantages, however, in that it is able to transport folded substrates, thus eliminating the potential for premature or incorrect folding. In certain embodiments, the engineered bacteria comprise a TAT or a TAT-like system and are capable of secreting the enzyme or portion thereof from the bacterial cytoplasm.

One of ordinary skill in the art would appreciate that the secretion systems disclosed herein may be modified to act in different species, strains, and subtypes of bacteria, and/or adapted to deliver different payloads.

### Kill Switch

Although the engineered microbes as described herein include the separated-enzyme approach to avoid a selective advantage and horizontal gene transfer, and although the engineered microbes are generally transient and do not colonize in the gastrointestinal tract, in some embodiments the inclusion of a kill switch can be contemplated. A kill switch is intended to actively kill engineered microbes in response to external stimuli. For example, the kill switch can be triggered by a particular factor in the environment that induces the production of toxic molecules within the microbe that cause cell death.

Bacteria engineered with kill switches have been engineered for in vitro research purposes, e.g., to limit the spread of a biofuel-producing microorganism outside of a laboratory environment. Bacteria engineered for in vivo administration to treat a disease or disorder can also be programmed to die at a specific time after the expression and delivery of a heterologous gene or genes, for example, a therapeutic gene(s), after the subject has experienced the therapeutic effect, or after the antibiotic is cleared from a subject. In some embodiments, the kill switch is activated to kill the bacteria after a period of time following the cessation of antibiotic administration and/or clearance of the antibiotic from the circulation etc in a subject. Alternatively, the bacteria may be engineered to die if they somehow spread outside of the gastrointestinal tract. Specifically, it can be useful to prevent long-term colonization of subjects by the microorganism, spread of the microorganism outside the area of interest (for example, outside the gut) within the subject, or spread of the microorganism outside of the subject into the environment (for example, spread to the environment through the stool of the subject). Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al., 2000), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al., 2010).

Kill-switches can be designed such that a toxin is produced in response to an environmental condition or external signal (e.g., the bacteria is killed in response to an external cue) or, alternatively designed such that a toxin is produced once an environmental condition no longer exists or an external signal is ceased.

In one embodiment, the bacterial toxin is bactericidal to the engineered bacterium. In one embodiment, the bacterial toxin is bacteriostatic to the engineered bacterium.

### Culture and Storage of Engineered Microorganisms

Cultures of engineered microorganisms can be incubated in nutrient medium under favorable conditions for a time sufficient to build biomass. For bacterial compositions for human use this is often at normal body temperature (37° C.), pH, and other parameters with values similar to the normal human niche. The environment can be actively controlled, passively controlled (e.g., via buffers), or allowed to drift. For example, for anaerobic bacterial compositions (e.g., gut microbiota), an anoxic/reducing environment can be employed. This can be accomplished by addition of reducing agents/factors such as cysteine to the broth, and/or stripping it of oxygen. As an example, a culture of a bacterial composition can be grown at 37° C., pH 7, in the medium above, pre-reduced with 1 g/L cysteine·HCl.

When the culture has generated sufficient biomass, it can be preserved for banking or storage. The engineered microorganisms can be placed into a chemical milieu that protects from freezing (adding ‘cryoprotectants’), drying (‘lyoprotectants’), and/or osmotic shock (‘osmoprotectants’), dispensing known amounts into multiple (optionally identical) containers and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term microorganism composition storage stability at temperatures elevated above cryogenic. If the bacterial composition comprises spore forming species and results in the production of spores, the final composition can be purified by additional means such as density gradient centrifugation and preserved using the techniques described above. As an example of cryopreservation, a composition culture can be harvested by centrifugation to pellet the cells from the culture medium, the supernatant decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Organism production can be conducted at larger scales of operation, especially for clinical development or commercial production. At larger scales, there can be several subcultivations of the engineered microorganisms prior to the final cultivation. At the end of cultivation, the culture is harvested to enable further formulation into a dosage form for administration. This can involve concentration, removal of undesirable medium components, and/or introduction into a chemical milieu that preserves the bacterial composition and renders it acceptable for administration via the chosen route. For example, a bacterial composition can be cultivated to a concentration of 10¹⁰ CFU/mL, then concentrated 20-fold by tangential flow microfiltration; the spent medium may be exchanged by diafiltering with a preservative medium consisting of 2% gelatin, 100 mM trehalose, and 10 mM sodium phosphate buffer. The suspension can then be freeze-dried to a powder and titrated.

After drying, the powder can be blended to an appropriate potency, and mixed with a filler such as microcrystalline cellulose for consistency and ease of handling, and the compositions formulated as provided herein.

### Administration and Efficacy

The engineered microorganisms as described herein can be administered to a subject by any suitable route of administration that permits the microorganism to enter the gastrointestinal tract (e.g., the small intestine). In some embodiments, following the administration of such compositions a therapeutic concentration of the expressed antibiotic-degrading enzyme (e.g., β-lactamase enzyme) is achieved and/or maintained in the tissues of the gastrointestinal tract (e.g., within the lumen of the gastrointestinal tract). In certain embodiments, the composition is combined with suitable excipients and formulated for enteral or rectal administration. General techniques applicable to the formulation and administration of the compositions comprising these engineered microbes can be found in “Remington's Pharmaceutical Sciences,” Mack Publishing Co., Easton, Pa., latest edition. The compositions described herein can also be administered or co-administered as part of a therapeutic regimen with other suitable therapeutic, prophylactic, or probiotic agents (e.g., administered concurrently or sequentially).

In embodiments where the compositions are administered to a subject orally, such compositions can be prepared or formulated as a food (e.g., a dairy product, preferably a fermented dairy product such as yogurt) or as a functional food (e.g., a nutritional supplement). In other embodiments, such compositions can be prepared or formulated, for example, as a pharmaceutical, a dietary supplement and/or a medical food.

In some embodiments, the compositions described herein comprise cells over a range of, for example, 2-5×10⁵, or more, e.g., 1×10⁶, 1×10⁷, 1×10⁸, 5×10⁸, 1×10⁹, 5×10⁹, 1×10¹⁰, 5×10¹⁰, 1×10¹¹ cells or more. The dosage range for the engineered microorganism depends upon the potency, and includes amounts large enough to produce the desired effect, e.g., reduction in gastrointestinal concentration of an antibiotic in a subject being treated with the antibiotic. The dosage should not be so large as to cause unacceptable adverse side effects. Generally, the dosage will vary with the type of illness, and with the age, condition, and sex of the patient. Where larger doses of the antibiotic are administered, it follows that the dosage of an engineered microbe as described herein can be increased. The dosage can be determined by one of skill in the art and can also be adjusted by the individual physician in the event of any complication.

For use in the various aspects described herein, an effective amount of engineered bacterial cells in a composition as described herein comprises at least 1×10⁵ bacterial cells, at least 1×10⁶ bacterial cells, at least 1×10⁷ bacterial cells, at least 1×10⁸ bacterial cells, at least 1×10⁹ bacterial cells, at least 1×10¹⁰ bacterial cells, at least 1×10¹¹ bacterial cells, at least 1×10¹² bacterial cells or more. In some embodiments of the aspects described herein, the bacterial cells are expanded or maintained in culture prior to administration to a subject in need thereof. In general, a single engineered microbe strain expressing antibiotic-degrading enzyme active on the antibiotic prescribed or being administered to the subject can be administered. However, where a subject is prescribed or administered a combination of two or more antibiotics, it can be appropriate to administer two or more engineered microbial strains, each expressing an antibiotic-degrading enzyme targeting a different one of the antibiotics prescribed or administered. In some embodiments, two or more bacterial strains are administered together, e.g., in a single admixture. However, it is specifically contemplated herein that two or more bacterial strains can be administered as separate dosage forms or sub-mixtures or sub-combinations of the strains.

In some embodiments, the compositions described herein can be administered in a form containing one or more pharmaceutically acceptable carriers. Suitable carriers are well known in the art and vary with the desired form and mode of administration of the composition. For example, pharmaceutically acceptable carriers can include diluents or excipients such as fillers, binders, wetting agents, disintegrators, surface-active agents, glidants, lubricants, and the like. The carrier can be a solid (including powder), liquid, or combinations thereof. Each carrier is preferably “acceptable” in the sense of being compatible with the other ingredients in the composition and not injurious to the subject. The carrier can be biologically acceptable and inert (e.g., it permits the composition to maintain viability of the biological material until delivered to the appropriate site). In addition to inert carriers, engineered microbes as described herein can optionally be formulated with one or more prebiotics to promote growth and metabolic activity of the engineered microbes at least during their transit of the gut.

Oral compositions can include an inert diluent or an edible carrier. For the purpose of oral therapeutic administration, the active compound can be incorporated with excipients and used in the form of tablets, lozenges, pastilles, troches, or capsules, e.g., gelatin capsules. Oral compositions can also be prepared by combining a composition of the present disclosure with a food. In some embodiments, the bacterium/bacteria or yeast can be formulated in a food item. Some non-limiting examples of food items to be used with the methods and compositions described herein include: popsicles, cheeses, creams, chocolates, milk, meat, drinks, pickled vegetables, kefir, miso, sauerkraut, etc. In other embodiments, the food items can be juices, refreshing beverages, tea beverages, drink preparations, jelly beverages, and functional beverages; alcoholic beverages such as beers; carbohydrate-containing foods such as rice food products, noodles, breads, and pastas; paste products such as fish, hams, sausages, paste products of seafood; retort pouch products such as curries, food dressed with a thick starchy sauce, and Chinese soups; dairy products such as milk, dairy beverages, ice creams, and yogurts; fermented products such as fermented soybean pastes, fermented beverages, and pickles; bean products; various confectionery products including biscuits, cookies, and the like, candies, chewing gums, gummies, cold desserts including jellies, cream caramels, and frozen desserts; instant foods such as instant soups and instant soy-bean soups; and the like. It is preferred that food preparations not require cooking after admixture with the engineered microorganisms to avoid killing the microbes.

In one embodiment a food used for administration is chilled, for instance, iced flavored water. In certain embodiments, the food item is not a potentially allergenic food item (e.g., not soy, wheat, peanut, tree nuts, dairy, eggs, shellfish or fish). Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, orange flavoring, or other suitable flavorings. These are for purposes of example only and are not intended to be limiting.

Formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Preparations for oral administration can be suitably formulated for slow release, controlled release, or sustained release of the genetically engineered bacteria described herein.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated.

In some embodiments, the pharmaceutically acceptable compositions are provided in single dosage forms. Single dosage forms can be in a liquid, suspension, or a solid form. Single dosage forms can be administered directly to a patient without modification or can be diluted or reconstituted prior to administration. In certain embodiments, a single dosage form can be administered in bolus form, e.g., single oral dose, including an oral dose that comprises multiple tablets, capsule, pills, etc.

In another embodiment, the composition can be delivered in a controlled release or sustained release system. In another embodiment, polymeric materials can be used to achieve controlled or sustained release of the engineered microorganisms described herein. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation can be inert, free of leachable impurities, stable on storage, sterile, and biodegradable.

The pharmaceutical compositions comprising engineered microorganisms as described herein can be packaged in a hermetically sealed container such as an ampoule or sachet indicating the quantity of the engineered microorganisms. In one embodiment, one or more of the pharmaceutical compositions is supplied as a dry lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions described herein is supplied as a dry lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Suitable bulking agents include glycine and arginine, either of which can be included at a concentration of 0-0.05%, and polysorbate-80 (optimally included at a concentration of 0.005-0.01%). Exemplary surfactants include but are not limited to polysorbate 20 and BRIJ surfactants.

The compositions described herein can also be prepared in the form of suppositories (e.g., with conventional suppository bases such as cocoa butter and other glycerides) or retention enemas for rectal delivery. Formulations suitable for rectal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, engineered microorganisms described herein can be formulated for colonic or rectal administration. The compositions can be prepared with carriers that will protect the bacteria against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from, for instance, Alza Corporation and Nova Pharmaceuticals, Inc. Liposomal suspensions can also be used as pharmaceutically acceptable carriers. These can be prepared according to methods known to those skilled in the art.

In some embodiments, an engineered microorganism composition can be encapsulated or microencapsulated (e.g., enteric-coated formulations). For instance, when the composition is to be administered orally, the dosage form is formulated so the composition is not exposed to conditions prevalent in the gastrointestinal tract before the small intestine, e.g., high acidity and digestive enzymes present in the stomach. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (EmCoat™ 120 N, Marcoat 125); cellulose acetate phthalate (trade names AQUACOAT™, AQUACOAT ECD™, SEPIFILM™, KLUCEL™, and ETOLOSE™); polyvinylacetate phthalate (trade name SURETERIC™); and methacrylic acid (trade name EUDRAGIT™). The encapsulation of compositions for therapeutic use is known in the art. Encapsulation can include hard-shelled capsules, which can be used for dry, powdered ingredients, or soft-shelled capsules. Capsules can be made from aqueous solutions of gelling agents such as animal protein (e.g., gelatin), plant polysaccharides or derivatives like carrageenans and modified forms of starch and cellulose. Other ingredients can be added to a gelling agent solution such as plasticizers (e.g., glycerin and or sorbitol), coloring agents, preservatives, disintegrants, lubricants and surface treatment.

In some embodiments, the engineered microorganisms are enterically coated for release into the gut or a particular region of the gut, for example, the small intestine. The typical pH profile from the stomach to the colon is about 1-4 (stomach), 5.5-6 (duodenum), 7.3-8.0 (ileum), and 5.5-6.5 (colon). In some diseases, the pH profile can be modified. In some embodiments, the coating is degraded in specific pH environments in order to specify the site of release. In some embodiments, at least two coatings are used. In some embodiments, the outside coating and the inside coating are degraded at different pH levels. In some embodiments, the enteric coated composition comprising engineered microorganisms further comprises a prebiotic.

Formulations of a yeast or bacterial composition can be prepared by any suitable method, typically by uniformly and intimately admixing the bacterial cells with liquids or finely divided solid carriers or both, in the required proportions and then, if necessary, shaping the resulting mixture into the desired shape. In some embodiments, the yeast or bacterial strain(s) as described herein is/are combined with one or more additional probiotic organisms prior to treatment of a subject. As used herein, the term “probiotic” refers to microorganisms that form at least a part of the transient or endogenous flora and thereby exhibit a beneficial prophylactic and/or therapeutic effect on the host organism. Probiotics are non-pathogenic under normal circumstances and include, but are not limited to, those designated “Generally Regarded as Safe (GRAS)” by the U.S. Food & Drug Administration.

A nutrient supplement comprising the engineered microorganisms as described herein can include any of a variety of nutritional agents, including vitamins, minerals, essential and nonessential amino acids, carbohydrates, lipids, foodstuffs, dietary supplements, short chain fatty acids and the like. Preferred compositions comprise vitamins and/or minerals in any combination. Vitamins for use in a composition as described herein can include vitamins B, C, D, E, folic acid, K, niacin, and like vitamins. The composition can contain any or a variety of vitamins as may be deemed useful for a particular application, and therefore, the vitamin content is not to be construed as limiting. Typical vitamins are those, for example, recommended for daily consumption and in the recommended daily amount (RDA), although precise amounts can vary. The composition can preferably include a complex of the RDA vitamins, minerals and trace minerals as well as those nutrients that have no established RDA, but have a beneficial role in healthy human or mammalian physiology. The preferred mineral format can include, for example, those that are in either the gluconate or citrate form which are more readily metabolized by lactic acid bacteria. Similar considerations can be employed to favor other classes of bacteria as needed.

The compositions as described herein can comprise from about 100 mg to about 100 g, alternatively from about 500 mg to about 50 g, and alternatively from about 1 g to about 40 g, of a prebiotic, per day or on a less than daily schedule.

If a patient has been indicated for treatment with an antibiotic (e.g., has an active bacterial infection or is indicated for prophylactic antibiotic use), in one embodiment the engineered microorganisms can be administered before the onset of treatment with the antibiotic to ensure maximum protection of the commensal flora in the gut. In one embodiment, the antibiotic may be discontinued 1, 2, or 3 days before the administration of, or clearance of, the engineered microorganisms is ceased to permit time for degradation of existing antibiotic to be metabolized following the last dose of antibiotic. In one embodiment, the antibiotic can be discontinued 3, 4, 5, 6, or 7 antibiotic half-lives before ceasing administration of the bacterial or yeast composition. As will be appreciated by those of skill in the art, the timing of administration of the engineered microorganism or composition thereof is different if the antibiotic route is parenteral vs oral. In typical practice, there will only be a few hours in between the prescription and beginning of the antibiotic course. Administration of the compositions as described herein preferably occurs prior to administration of the antibiotic to permit protection from the first dose. In addition, it is contemplated herein that the engineered microorganisms or compositions thereof are administered such that their presence is sustained through the entire course of antibiotics, and optionally beyond the last dose of the antibiotic until the antibiotic is effectively cleared.

Any of the preparations described herein can be administered once on a single occasion or on multiple occasions, such as once a day for several days or more than once a day on the day of administration (including twice daily, three times daily, or up to five times daily). Or the preparation can be administered intermittently according to a set schedule, e.g., once weekly, once monthly, or when the patient relapses from the primary illness. In another embodiment, the preparation can be administered on a long-term basis to assure the maintenance of a protective or therapeutic effect. Typically, administration of the engineered microorganisms will begin prior to onset of the treatment with the antibiotic (e.g., at least 2 hours prior, at least 3 hours prior, at least 6 hours prior, at least 12 hours prior, at least 24 hours prior) and will continue until administration of the antibiotic is ceased and/or the antibiotic is metabolized and cleared by the subject. The schedule and timing of the administration of the engineered microorganisms will depend on whether the organism is transient or engrafts; the clearance of transient organisms will also depend on the particular microorganism employed).

Thus, in one embodiment, the compositions described herein are administered at least 2 hours prior to administration of the first dose of antibiotic (e.g., at least 1.5 hours, at least 1 h, at least 45 min, at least 30 min, at least 15 min or at least 5 minutes prior to administration of the first dose of the antibiotic. In another embodiment, the compositions described herein are administered simultaneously with the first dose of antibiotic (preferably the antibiotic is administered by parenteral administration when simultaneous administration is indicated). In certain embodiments, administration of the engineered microorganisms is repeated at least once during the course of antibiotics (e.g., at least twice, at least 3 times, at least 4 times, at least 5 times, at least 6 times, at least 7 times, at least 10 times, at least 14 times, at least 21 times etc.). In one embodiment, the timing between two administered doses of the engineered microorganisms is less than 40 hours, less than 35 hours, less than 30 hours, less than 25 hours, less than 24 hours, less than 20 hours, less than 15 hours, less than 12 hours, less than 10 hours, less than 6 hours or less). In one embodiment, the maximum amount of time between doses comprises the measured or estimated time of transit through the colon for the treated subject.

Excluded Bacteria: As will be readily appreciated by one of skill in the art, a composition as described herein for administration to a subject will ideally not comprise one or more pathogenic bacteria. In one embodiment, a composition as described herein (e.g., a pharmaceutical or probiotic composition comprising engineered microorganisms) does not include an organism conventionally classified as a pathogenic or opportunistic organism. It is possible that a function shared by all members of a given taxonomic group could be beneficial, e.g., for providing particular metabolites, yet for other reasons the overall effect of one or more particular members of the group is not beneficial and is, for example, pathogenic. Clearly, members of a given taxonomic group that cause pathogenesis, e.g., acute gastrointestinal pathologies, are to be excluded from the therapeutic or preventive methods and compositions described herein.

In one embodiment, the bacterial composition does not comprise at least one of Acidaminococcus intestinalis, Lactobacillus casei, Lactobacillus paracasei, Raoultella sp., and Streptococcus mitis. In another embodiment, the bacterial composition does not comprise any of these.

In another embodiment, the bacterial composition does not comprise at least one of Barnesiella intestinihominis, Lactobacillus reuteri, Enterococcus hirae, Enterococcus faecium, or Enterococcus durans, Anaerostipes caccae, Clostridium indolis, Staphylococcus wameri, or Staphylococcus pasteuri, and Adlercreutzia equolifaciens. In another embodiment, the bacterial composition does not comprise any of these.

In another embodiment, the bacterial composition does not comprise at least one of Clostridium botulinum, Clostridium cadaveris, Clostridium chauvoei, Clostridium clostridioforme, Clostridium cochlearium, Clostridium difficile, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium septicum, Clostridium sordellii, Clostridium sphenoides, and Clostridium tetani. In another embodiment, the bacterial composition does not comprise any of these.

In another embodiment, the bacterial composition does not comprise at least one of Escherichia coli and Lactobacillus johnsonii. In another embodiment, the bacterial composition does not comprise any of these.

In another embodiment, the bacterial composition does not comprise at least one of Clostridium innocuum, Clostridium butyricum, and Blautia producta (previously known as Peptostreptococcus productus). In another embodiment, the bacterial composition does not comprise any ofthese.

In another embodiment, the bacterial composition does not comprise at least one of Eubacteria, Fusobacteria, Propionibacteria, Escherichia coli, and Gemmiger.

In another embodiment, the compositions described herein do not comprise pathogenic bacteria in the Genera Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Chlamydia, Chlamydophila, Campylobacter, Burkholderia, Brucella, Borrelia, Bordetella, Bacillus, multi-drug resistant bacteria, extended spectrum A-lactam resistant Enterococci (ESBL), Carbapenem-resistant Enterobacteriaceae (CRE), or vancomycin-resistant Enterococci (VRE).

In other embodiments, the compositions described herein do not comprise pathogenic species or strains, such as Aeromonas hydrophila, Campylobacter fetus, Plesiomonas shigelloides, Bacillus cereus, Campylobacter jejuni, enteroaggregative Escherichia coli, enterohemorrhagic Escherichia coli, enteroinvasive Escherichia coli, enterotoxigenic Escherichia coli (such as, but not limited to, LT and/or ST), Escherichia coli 0157:H7, Helicobacter pylori, Klebsiellia pneumonia, Lysteria monocytogenes, Plesiomonas shigelloides, Salmonella spp., Salmonella typhi, Salmonella paratyphi, Shigella spp., Staphylococcus spp., Staphylococcus aureus, vancomycin-resistant enterococcus spp., Vibrio spp., Vibrio cholerae, Vibrio parahaemolyticus, Vibrio vulnifcus, or Yersinia enterocolitica.

In one embodiment, the bacterial compositions or formulations as described herein do not comprise Klebsiella pneumoniae, Proteus mirabilis, Enterobacter cloacae, or Bilophila wadsworthia.

### Efficacy Measurement

The term “effective amount” as used herein refers to the amount of a population of engineered bacterial or yeast cells needed to locally degrade at least 50% of an administered antibiotic in the gastrointestinal tract (i.e., at least 60%, at least 70%, at least 80%, at least 90%, at least 95%, at least 99%, or even 100% (below detectable levels) of the antibiotic). Alternatively, the effective amount refers to the amount of a population of engineered bacterial or yeast cells needed to maintain the commensal bacteria in the gut (e.g., to preserve diversity or volume of the commensal bacteria). An effective amount as used herein also includes an amount sufficient to prevent or delay the development of a symptom of a disease (e.g., C. difficile infection), alter the course of a symptom of the disease (for example, reducing diarrhea), or reverse a symptom of the disease. It is understood that for any given case, an appropriate “effective amount” can be determined by one of ordinary skill in the art using routine experimentation. Given the intricacies of the body and the nature of cell establishment, the “effective amount” of cells may vary among different patients, however one can easily determine in hindsight if the amount of cells administered was indeed an ‘effective amount.” Thus, further treatments can be modified accordingly. Note that long-term colonization or establishment, while often desirable, is not necessary for effective treatment as regular administration can achieve effective treatment as well.

Where a loss of species diversity is generally the trigger for opportunistic infection, e.g., productive or symptomatic infection with C. difficile, one measure of efficacy of treatment with an engineered microbe as described herein is maintenance of gut microbial diversity in a subject receiving antibiotic treatment to an extent greater than normally occurs upon treatment with that antibiotic in the absence of an engineered microbe as described herein. There are a number of different measures of microbial diversity, but they commonly look at two parameters for a community: the number of different microbes, e.g., gut bacteria, in a sample, and the relative abundance of the different microbes. A significant change in either of these parameters can change the ability of the community to suppress or resist overgrowth or over-activity of a given species, such as C. difficile. While other indices can be used, the Shannon diversity index is commonly used to express gut microbiota diversity. In one embodiment, then, an effective treatment is one that maintains the microbial diversity of the gut of a given subject, as measured by the Shannon diversity index, upon antibiotic treatment, or throughout a course of antibiotic treatment for an infection.

One of skill in the art will appreciate that there is a great degree of variance of diversity status among individuals, thus, in highly diverse individuals, a reduction of less than 50% of the starting diversity can still permit colonization resistance to pathogenic bacteria. In subjects initially having lower diversity, a smaller decrease in diversity could have greater physiological effects.

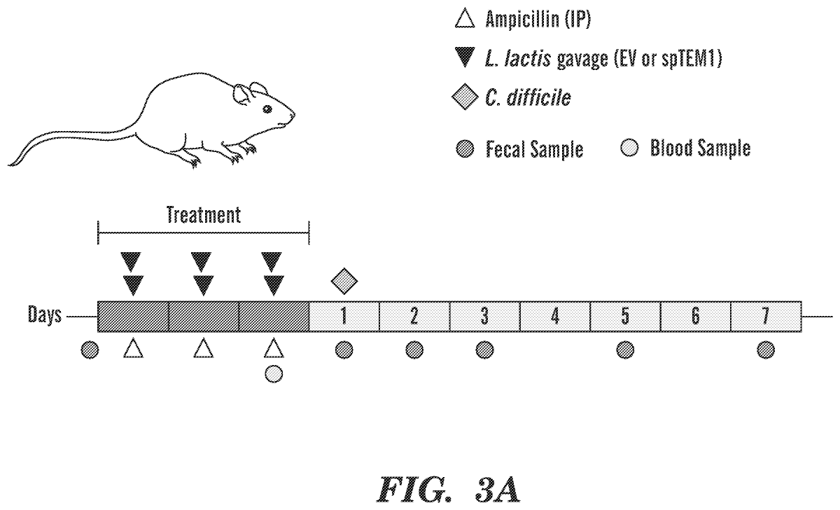

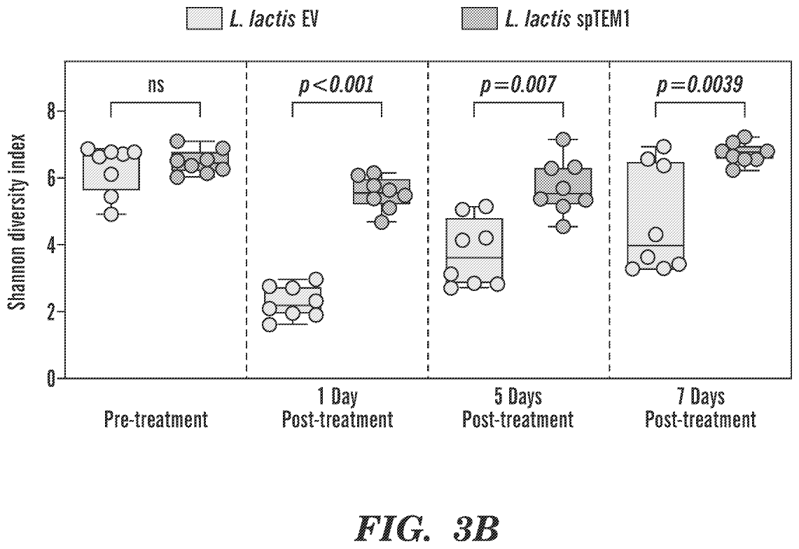

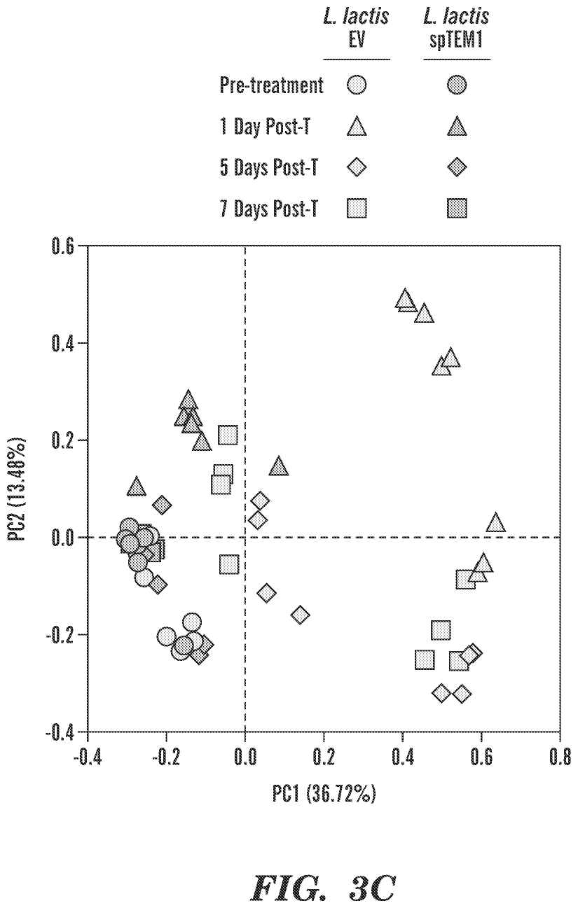

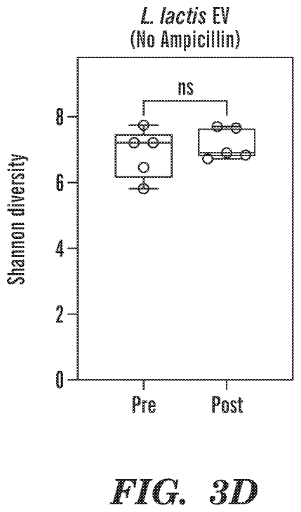

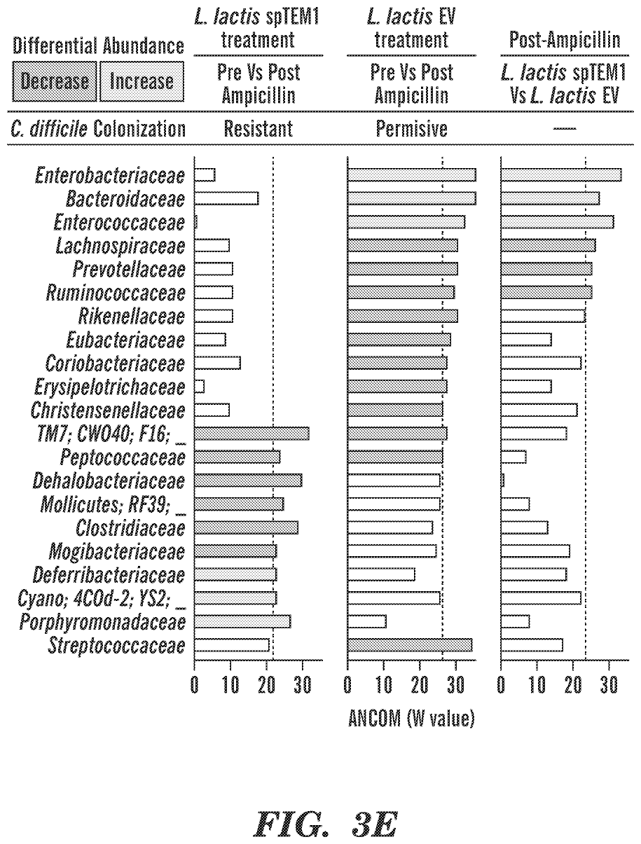

Alternatively, one of skill in the art can use the efficacy measurement described in the working Examples (see e.g., FIG. 3D). Assuming that the initial status of the microbiota is 100% effective at excluding C. difficile, sufficient efficacy can be assessed as a combination of (i) smaller reduction in the microbial diversity, (ii) conservation of the initial composition of the microbiota (see e.g., FIG. 3C) and (iii) preservation of bacterial families that are known to have effects on colonization resistance, in particular Lachnospiraceae, and Ruminococcaceae. In one embodiment, efficacious treatment is determined by scoring an individual's microbial diversity as “no change in score relative to initiation of treatment” or “minimal change in score relative to initiation of treatment.” By “minimal” in this context is meant, for example, less than 5% change, e.g., less than 4% change, less than 3% change, less than 2% change, or less than 1% change.

In one embodiment, effective treatment is determined by a reduction in one or more symptoms associated with an opportunistic bacterial infection in the gastrointestinal tract.

In some embodiments, the subject is further evaluated using one or more additional diagnostic procedures, for example, by medical imaging, physical exam, laboratory test(s), clinical history, family history, genetic test, and the like. Medical imaging is well known in the art. As such, the medical imaging can be selected from any known method of imaging, including, but not limited to, ultrasound, computed tomography scan, positron emission tomography, photon emission computerized tomography, and magnetic resonance imaging.

The invention may be as described in any one of the following numbered paragraphs:
1. A composition comprising a microorganism engineered to degrade an antibiotic in the mammalian gut, wherein the microorganism is also engineered to reduce the likelihood of horizontal transmission of its engineered antibiotic-degrading capacity.
2. The composition of paragraph 1, wherein the microorganism's engineered antibiotic degrading capacity comprises expression and secretion of an enzyme activity that degrades the antibiotic.
3. The composition of paragraph 2, wherein the enzyme is encoded in first and second parts, on separate, first and second nucleic acid constructs, wherein neither construct on its own encodes active antibiotic-degrading enzyme, and where both parts of the enzyme are needed to provide antibiotic-degrading activity, thereby reducing the likelihood of horizontal transmission of the engineered antibiotic-degrading activity.
4. The composition of paragraph 1, wherein the microorganism is a bacterium or a yeast.
5. The composition of paragraph 3 or paragraph 4, wherein the first and second constructs encode the first and second parts of the enzyme as first and second fusion polypeptides, each comprising a respective member of a specific binding pair.
6. The composition of paragraph 5, wherein the first and second fusion polypeptides are secreted by the microorganism into its surrounding environment.
7. The composition of paragraph 5 or paragraph 6, wherein binding of the first and second fusion polypeptides via the respective members of the specific binding pair promotes the physical interaction of the first and second parts of the enzyme and reconstitution of antibiotic-degrading enzymatic activity.
8. The composition of any one of paragraphs 5-7, wherein the respective members of the specific binding pair promote covalent bonding between the first and second fusion polypeptides.
9. The composition of any one of paragraphs 1-8, wherein the antibiotic-degrading activity comprises β-lactamase enzyme activity, Staphylococcus aureus mph(C) gene enzyme activity, S. aureus lincosamide nucleotidyltransferase lnu(A) gene enzyme activity, an Enterococcus faecium lnu(b) gene enzyme activity or an Escherichia coli ereB gene enzyme activity.
10. The composition of paragraph 9, wherein the β lactamase is a TEM1 β lactamase.
11. The composition of paragraph 10, wherein the TEM1 β lactamase is encoded in first and second parts, on separate, first and second nucleic acid constructs, wherein the first nucleic acid construct encodes β lactamase fragment (BLF) 1, comprising SEQ ID NO: 1 or 3, and the second nucleic acid construct encodes BLF 2, comprising SEQ ID NO: 5 or 7.
12. The composition of any one of paragraphs 3-11, wherein the first and second constructs encode the first and second parts of the enzyme as first and second fusion polypeptides, respectively, each comprising a respective member of the SpyTag/SpyCatcher specific binding pair.
13. The composition of any one of paragraphs 1-12, wherein the microorganism is an engineered generally regarded as safe (GRAS) microorganism.
14. The composition of any one of paragraphs 1-13, wherein the microorganism is an engineered lactic acid bacterium.
15. The composition of any one of paragraphs 1-14, wherein the microorganism is an engineered Lactococcus lactis bacterium.
16. The composition of any one of paragraphs 1-15, in a formulation for oral delivery.
17. A viable lyophilized microorganism as recited in of any one of paragraphs 1-16.
18. The composition of any one of paragraphs 1-17, formulated as a pill, tablet or capsule.
19. A method of treating a bacterial infection, the method comprising administering an antibiotic and a composition of any one of paragraphs 1-18.
20. The method of paragraph 19, wherein the antibiotic is delivered parenterally or orally.
21. The method of paragraph 19 or paragraph 20, wherein the antibiotic is delivered intravenously.
22. The method of any one of paragraphs 19-21, wherein the composition is administered before the antibiotic is administered.
23. The method of any one of paragraphs 19-22, wherein the antibiotic is delivered parenterally, and the composition is administered before or at the same time the antibiotic is delivered.
24. The method of any one of paragraphs 19-23, wherein the composition is administered orally.
25. The method of paragraph 19 or paragraph 20, wherein the composition is orally administered before the antibiotic is orally administered.
26. The method of any one of paragraphs 19-25 wherein the antibiotic is a β-lactam antibiotic, and the microorganism in the composition is engineered to express a β-lactamase enzyme.
27. The method of any one of paragraphs 19-26, wherein the antibiotic is selected from penicillin, penicillin G, penicillin V, oxacillin, nafcillin, dicloxacillin, amoxicillin, ampicillin, ticarcillin, piperacillin, cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, ceftobiprole, meropenem, doripenem, ertapenam, or aztreonam).
28. The method of any one of paragraphs 19-27, wherein the microorganism in the composition promotes the degradation of the antibiotic in the gut, thereby limiting or preventing antibiotic-induced gut dysbiosis.
29. A method of preventing or limiting an antibiotic-induced dysbiosis in a subject in need of antibiotic administration, the method comprising administering an antibiotic and a composition of any one of paragraphs 1-18.
30. The method of paragraph 29, wherein the composition is administered orally.
31. The method of paragraph 29 or 30, wherein the antibiotic is administered parenterally or orally.
32. The method of any one of paragraphs 29-31, wherein the composition is orally administered before the antibiotic is orally administered.
33. The method of any one of paragraphs 29-32, wherein the bacterial infection is an infection with a bacterium sensitive to a β-lactam antibiotic, and the antibiotic is a β-lactam antibiotic.
34. The method of any one of paragraphs 29-33, wherein the antibiotic is selected from penicillin, penicillin G, penicillin V, oxacillin, nafcillin, dicloxacillin, amoxicillin, ampicillin, ticarcillin, piperacillin, cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, ceftobiprole, meropenem, doripenem, ertapenam, or aztreonam).
35. A method of preventing C. difficile pathology in a subject treated with an antibiotic, the method comprising administering a composition of any one of paragraphs 1-18 to the subject.
36. The method of paragraph 35, wherein the composition is administered orally.
37. The method of paragraph 35 or 36, wherein the antibiotic is administered parenterally or orally.
38. The method of any one of paragraphs 35-37, wherein the composition is orally administered before the antibiotic is orally administered.
39. The method of any one of paragraphs 35-38, wherein the subject comprises a bacterial infection comprising a bacterium sensitive to a β-lactam antibiotic, and the antibiotic is a 3-lactam antibiotic.
40. The method of any one of paragraphs 35-39, wherein the antibiotic is selected from penicillin, penicillin G, penicillin V, oxacillin, nafcillin, dicloxacillin, amoxicillin, ampicillin, ticarcillin, piperacillin, cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, ceftobiprole, meropenem, doripenem, ertapenam, or aztreonam.
41. A system for limiting or preventing antibiotic-induced dysbiosis, the system comprising: (i) a first nucleic acid construct, encoding a first fusion polypeptide comprising a first part of an antibiotic-degrading enzyme, fused to a first member of a specific binding pair, operably linked to sequence permitting expression of the first fusion polypeptide; (ii) a second nucleic acid construct, encoding a second fusion polypeptide comprising a second part of an antibiotic-degrading enzyme, fused to the second member of the specific binding pair, operably linked to sequence permitting expression of the second fusion polypeptide, wherein neither the first fusion polypeptide nor the second fusion polypeptide alone can degrade antibiotic, but wherein a physical association between the first and second fusion polypeptides permits association between the first and second parts of the antibiotic-degrading enzyme to form an active antibiotic-degrading complex.
42. The system of paragraph 41, comprised by a microorganism.
43. The system of paragraph 41, wherein the microorganism is a bacterium or a yeast.
44. The system of any one of paragraphs 41-43, wherein the respective members of the specific binding pair promote covalent bonding between the first and second fusion polypeptides.
45. The system of any one of paragraphs 41-44, wherein the antibiotic-degrading activity comprises a R lactamase enzyme activity, Staphylococcus aureus mph(C) gene enzyme activity, S. aureus lincosamide nucleotidyltransferase lnu(A) gene enzyme activity, an Enterococcus faecium lnu(b) gene enzyme activity or an Escherichia coli ereB gene enzyme activity.
46. The system of paragraph 45, wherein the β lactamase is a TEM1 β lactamase.
47. The system of paragraph 41, wherein the first nucleic acid construct comprises sequence encoding β lactamase fragment (BLF) 1, comprising SEQ ID NO: 1 or 3, and the second nucleic acid construct comprises sequence encoding BLF 2, comprising SEQ ID NO: 5 or 7.
48. The system of any one of paragraphs 41-47, wherein the specific binding pair is the respective members of the SpyTag/SpyCatcher specific binding pair.
49. The system of any one of paragraphs 41-48, wherein the microorganism is an engineered GRAS microorganism.
50. The system of any one of paragraphs 41-49, wherein the microorganism is a lactic acid bacterium.
51. The system of any one of paragraphs 41-50, wherein the microorganism is a Lactococcus lactis bacterium.

### Examples

### Example 1

Study Aims: Antibiotics are life-saving medicines, yet their use negatively impacts the healthy gut microbiota. Most notably antibiotic use in hospitalized patients represents the highest risk factor for contracting Clostridioides difficile. To prevent collateral damage by antibiotics in the gut microbiota, an engineered L. lactis strain that is able to degrade β-lactam antibiotics was generated by encoding a gene trait that is impervious to dissemination by horizontal gene transfer. The efficacy of the engineered probiotic in preventing dysbiosis was tested in a mouse model that recapitulates the infection of C. difficile upon a course of parenteral ampicillin. Oral administration of the engineered probiotic to mice that received parenteral ampicillin was successful in preventing gut dysbiosis on three key aspects. First, treatment with the engineered probiotic significantly diminished the loss of species diversity within the gut and enabled a fast recovery of the original bacterial population structure following ampicillin administration. Second, there was a significant reduction in the magnitude and duration of antimicrobial resistance gene (ARG) selection in the mice treated with the engineered probiotic compared to mice treated with the control probiotic. Lastly, the engineered probiotic protected the native microbiota to an extent enough to maintain the colonization resistance against C. difficile in 100% of the treated mice. Importantly, the probiotic treatment did not affect the concentration of ampicillin in serum, which ensures that the antibiotic will remain effective in clearing infections outside the gut. The engineered probiotics described herein can be used to avoid antibiotic-induced dysbiosis, prevent the enrichment of ARG in the gut and preclude the loss of colonization resistance against intestinal pathogens.

### INTRODUCTION

Disruption of the ecological balance in healthy gut microbial communities, termed “dysbiosis,” has been associated with a wide range of immunological, metabolic and neurologic disorders such as allergies, autoimmunity, obesity, and autism among others.¹ Antibiotic therapy is essential for treating bacterial infections; however, its use often induces dysbiosis because systemically circulating antibiotics reach the gut via biliary excretion.²,³ Since antibiotic presence in the gut is only required when treating gastro-intestinal infections, all other antibiotic usage indications should exclude antibiotics from the gut to spare the gut microbiota. In particular, disturbance of the commensal microbial communities in the gut also increases the risk of secondary infections due to the loss of colonization resistance.¹ Most notably, repeated exposure of the gut microbiota to antibiotics eliminates commensal bacteria from their intestinal niche, opening the opportunity for pathogenic microorganisms, such as Clostridioides difficile, to colonize and proliferate. In their 2019 Antibiotic Resistance Threats Report, the Centers for Disease Control designated the highest threat level to C. difficile infection, which causes ˜224,000 cases and ˜13,000 deaths per year. Exposure to antibiotics also contributes to the emergence of antimicrobial resistance through the enrichment of gut bacterial populations that carry antimicrobial resistance genes (ARG),⁴,⁵ which in turn can be transferred to pathogenic bacteria through horizontal gene transfer (HGT).⁶ Therefore, this study aims to satisfy the pressing need for interventions that protect the healthy gut microbiota while permitting antibiotics to clear infecting pathogenic bacteria at other locations in the body.

The global yearly usage of antibiotics is ˜77 billion doses, of which ˜48 billion doses correspond to β-lactams,⁷ the antibiotic group this work focuses on. The β-lactam antibiotics (penicillins, carbapenems and cephalosporins) are highly effective for treating bacterial infections and represent one third of the antibiotics in the market. In the US, ˜15 million patients receive intravenous (IV) β-lactam antibiotics every year. Clinicians often prescribe probiotics during antibiotic treatment as a means for avoiding common side effects. A meta-analysis of 39 human randomized clinical trials with nearly 10,000 patients demonstrated that probiotics are moderately effective in preventing C. difficile-associated diarrhea.⁸ However, this beneficial effect is likely to be the result of a passive protection mechanism that does not prevent the reduction of the diversity of native bacterial species, often resulting in mixed clinical outcomes.⁸ Building on this baseline beneficial effect and leveraging the utility of probiotics as delivery vectors of biologics to the intestine, the inventors set out to create an engineered probiotic formulation that when taken simultaneously with an IV β-lactam course might act as a preventative intervention that minimizes dysbiosis, precludes enrichment of antimicrobial resistance determinants and reduces the risk of acquiring secondary bacterial infections.

Bacteria have evolved different mechanisms to survive the action of β-lactam antibiotics that include the production of lactam ring-hydrolyzing enzymes, modification of the cell wall target, efflux pumps, among others.^{9,10} The most common resistance mechanism is the production of β-lactamases, which are enzymes that act on the bacterial cell wall to inactivate incoming β-lactam antibiotics.¹¹ The expression of these enzymes by pathogenic bacteria greatly increases their fitness under antibiotic pressure and represents a growing challenge for the treatment of infections.¹² However, if repurposed through synthetic biology approaches, bacterial expression of β-lactamases could represent a novel strategy to eliminate antibiotics from unwanted locations in the body. It was hypothesized that transient gut colonization by a probiotic population that secretes a β-lactamase as a public good can prevent the collapse of the gut microbial communities despite the presence of a β-lactam antibiotic. Two conditions are needed to make a safe β-lactamase-expressing probiotic. First, the antibiotic degradation trait should not be amenable for HGT to other bacteria. Second, the trait cannot confer to the producing cell a selective advantage over the native microbial communities in order to avoid overgrowth of the probiotic in the gut or in the environment. Presented herein is the construction and evaluation of an engineered Lactococcus lactis strain that prevents antibiotic-induced dysbiosis, thwarts ARG enrichment and maintains colonization resistance against C. difficile in a mouse model.

### Results

Engineering an extracellular heterodimeric β-lactamase expression system. L. lactis was selected as a probiotic “chassis” as it displays ecological and safety features that are suitable for this application. L. lactis is a Gram-positive bacterium that is usually found in fermented milk products and has been safely consumed in high concentrations for millennia.¹³ Importantly, L. lactis is a probiotic that does not colonize the human gut nor affects the composition of the microbiota when ingested.¹⁴ Yet, it is metabolically active during its transit through the intestines and thus represents an ideal platform for the transient delivery of biologics to the gut. In addition, L. lactis is considered a Generally Regarded as Safe (GRAS) organism and has been safely used as a drug delivery vector in multiple clinical trials.^{15,16}

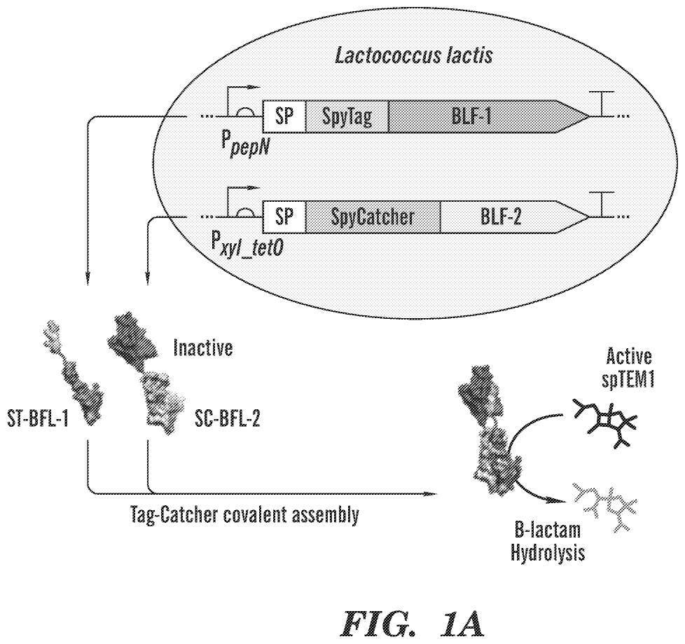

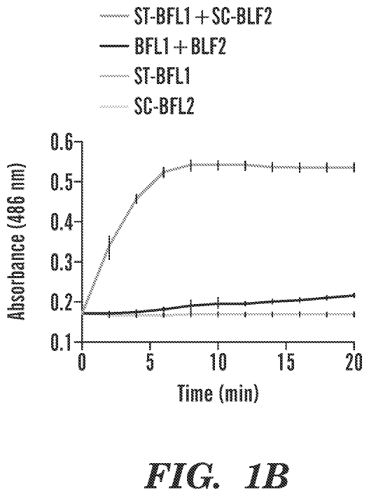

A L. lactis strain was generated that is able to inactivate β-lactam antibiotics in its surroundings by secreting a split enzyme that is encoded in a two-gene biosynthesis pathway. By splitting β-lactam degradation into a genetically unlinked trait the selective advantage conferred upon a single locus was removed, thereby limiting the possible spread of the engineered trait in the population through a single HGT event. To achieve this, a heterodimeric version of the TEM1 β-lactamase (spTEM1) that regains its activity when reconstituted in the extracellular environment was generated. It has been demonstrated that the TEM1 β-lactamase polypeptide can be divided into two enzymatically inactive fragments (BLF1 and BLF2) that contain no periodic secondary structure and that when brought into close proximity undergo protein re-folding, restoring hydrolytic activity.¹⁷ To prevent heterodimer dissociation and enhance the rate at which the active enzyme is reconstituted, the inventors fused the BLF1 and BLF2 fragments to the covalent bond-forming cognate domains SpyTag (ST) and SpyCatcher (SC), respectively.¹⁸ These parts are cloned into the L. lactis spTEM1 strain that constitutively expresses the subunit fragments from independent genetic loci and secretes them via the usp45 signal peptide for extracellular assembly (FIG. 1A). The colorimetric substrate nitrocefin was used to evaluate the β-lactamase activity in culture supernatants and it was determined that the ST-BLF-1 and the SC-BLF-2 subunits alone do not display β-lactamase activity and that in the absence of the covalent bond afforded by the ST and SC interaction, the BLF1 and BLF2 fragments are slow in reconstituting and show minimal β-lactamase activity (FIG. 1B). These results indicate that posttranslational assembly of heterodimeric fragments of the TEM1 β-lactamase enables the reconstitution of enzymatic activity and allows the system to be recoded into independent genetic loci.

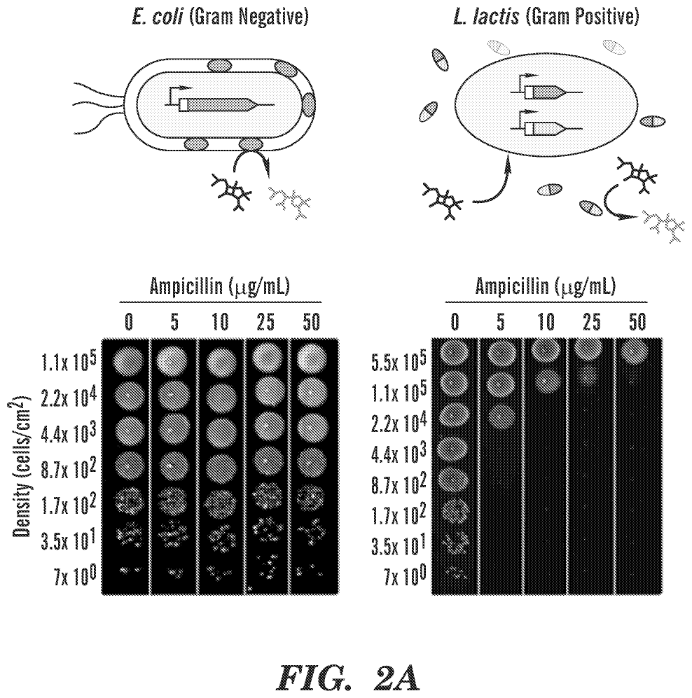

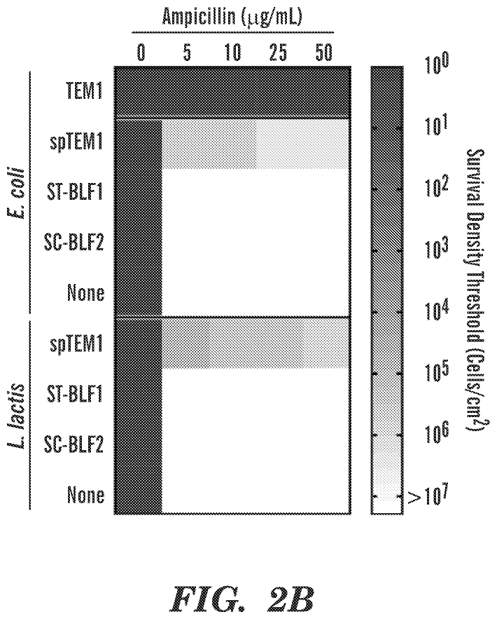

The spTEM1 β-lactamase system does not confer ampicillin resistance to producer cells. β-lactamases confer a strong fitness advantage to the producing cell by cleaving the β-lactam ring upon the arrival of the antibiotic to the cell wall. In Gram-negative bacteria, β-lactamases are actively exported to the periplasmic space, which increases their local concentration near the cell-wall and allows high levels of resistance.¹² In Gram-positive bacteria, although β-lactamase expression is not the principal mechanism for β-lactam resistance, β-lactamase-producing species have evolved lipoprotein anchors that keep the enzyme bound to the cell wall.^{9,19} Since subcellular localization near the cell wall is a trait that has been strongly selected during the evolution of β-lactamases, it was hypothesized that the secretion and extracellular reconstitution features engineered into the L. lactis spTEM1 strain prevent a selective advantage on the producer cell as the enzyme is free to diffuse away from the cell wall, leaving it unprotected from the antibiotic. To test this, the effect of the spTEM1 expression and cell density on the ability to survive different concentrations of ampicillin was evaluated. It was found that expression of the spTEM1 β-lactamase system does not confer ampicillin resistance to single cells of L. lactis (FIG. 2A). In E. coli, periplasmic localization of TEM1 β-lactamase enables the growth in ampicillin as single colonies. In contrast, to survive the action of ampicillin the L. lactis spTEM1 strain requires a threshold cell density at which the concentration of extracellular β-lactamase is enough to inactivate the ampicillin in the occupied area. Below this survival density threshold, the entire population collapses before the antibiotic can be inactivated (FIG. 2A). Therefore, expression of the spTEM1 β-lactamase system in L. lactis does not confer a selective advantage to single cells but rather confers the ability to survive ampicillin as an emergent property that depends on population density. These results demonstrate that β-lactamase expression can be engineered as a microbial public good with no benefit to the producer cell, which in itself constitutes a biosafety mechanism that precludes the engineered probiotic from gaining a competitive advantage over native bacteria.

The spTEM1 β-lactamase system is not amenable to HGT. Stable acquisition of a new genetic trait by HGT requires both the physical mobilization of DNA into the recipient cell and that the expression of the newly obtained genetic material provides enough selective advantage to outcompete the rest of the population. While encoding the spTEM1 β-lactamase system in separate genetic loci reduces the likelihood of transfer to a single recipient cell, the scenario of simultaneous acquisition of the two β-lactamase fragments cannot be completely ruled out. The inventors investigated whether stable acquisition of the spTEM1 β-lactamase genes to a Gram-negative bacterium confers a fitness advantage in the presence of ampicillin. For this, E. coli strains that constitutively express either or both fragments of the spTEM1 β-lactamase system were constructed and it was found that, even in a bacterium with a periplasm, the expression of the spTEM1 β-lactamase genes is not sufficient to confer resistance to ampicillin (FIG. 2B). Similar to L. lactis spTEM1, E. coli cells expressing the spTEM1 (3-lactamase system require a threshold cell density to allow for population-level survival and no growth as single colonies is evidenced. Since the secretion signals used in the spTEM1 β-lactamase system are designed to work with the L. lactis machinery, there could be a transport-related incompatibility that keeps the enzyme from actively reaching the cell wall of E. coli. Consequently, even in the scenario where both genes of the spTEM1 β-lactamase system get transferred to a Gram-negative recipient cell, it cannot provide sufficient protection to endow it with ampicillin resistance and thus it is unlikely to be evolutionarily selected. These results demonstrate that the engineered β-lactam degradation trait is not amenable to HGT as a result of the combination of reducing the likelihood of physical transfer of two independent DNA molecules and the absence of a selective advantage to the potential recipient cell.

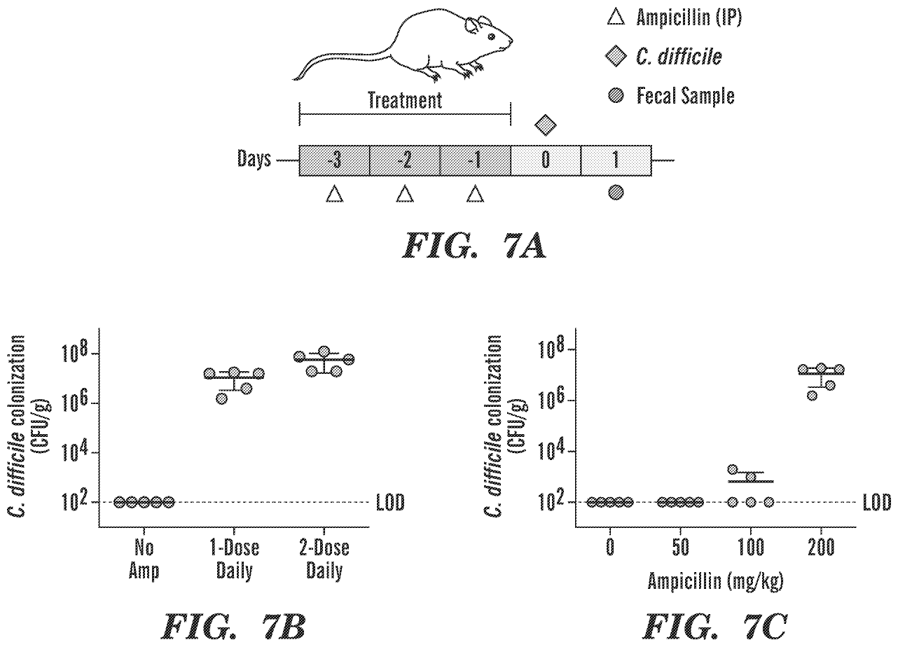

L. lactis spTEM1 protects the diversity and composition of gut the microbiota in an ampicillin-induced dysbiosis murine model. To test the efficacy of L. lactis spTEM1 in preventing ampicillin-induced dysbiosis, a mouse model was developed that utilizes susceptibility to C. difficile infection as an endpoint indication of gut dysbiosis. It was determined that a daily dose of 200 mg/kg of intraperitoneal ampicillin for 3 days is sufficient to abrogate colonization resistance and sensitize the mouse gut to oral infection with C. difficile spores (FIGS. 7A-7C). Based on this antibiotic regimen, the inventors devised a probiotic intervention aimed at populating the mouse gut with L. lactis spTEM1 while ampicillin circulates in the body (FIG. 3A). To ensure a robust presence of the probiotic in the gut, two oral doses of 10¹⁰ CFU of L. lactis were administered 2 hours before and simultaneous with the ampicillin injection, via oral gavage. The inventors used 16S rDNA sequencing to characterize the composition of the native bacterial communities in ampicillin-treated mice that received either the L. lactis spTEM1 strain or the control strain L. lactis EV, a strain that carries an empty expression vector. It was found that populating the mouse gut with the L. lactis spTEM1 strain significantly reduced the impact of ampicillin on the bacterial alpha-diversity compared to the intervention with the L. lactis EV strain (FIG. 3B). After the 3-day ampicillin treatment, the group that received the L. lactis EV strain suffered a sharp drop in their bacterial diversity, from which 62.5% of the mice were not able to recover to their pre-treatment diversity levels by day 7 of the last ampicillin dose. In contrast, mice that received the L. lactis spTEM1 strain displayed a significantly smaller decrease in their Shannon diversity index and fully recovered to their original bacterial diversity values (FIG. 3B).

The inventors further investigated the effect of the L. lactis spTEM1 strain in preventing changes in the taxonomic profile of the microbiota upon treatment with ampicillin. Principal Coordinate Analysis (PCoA) of the beta-diversity revealed that the composition of gut microbiota of mice receiving the L. lactis spTEM1 deviated slightly at day 1 after the ampicillin treatment before returning to its original composition as evidenced in the close clustering with pre-treatment samples (FIG. 3B). On the other hand, the gut microbiota of mice dosed with the L. lactis EV strain suffered large changes in their composition that made them diverge from its original state and could not be reestablished even after 7 days of the last ampicillin dose (FIG. 3B). These results demonstrate that transient colonization of the murine gut by an extracellular β-lactamase-expressing probiotic significantly diminishes the loss of bacterial diversity and prevents changes in the composition of the gut microbial communities after the use parenteral ampicillin.

L. lactis spTEM1 prevents the enrichment of ARG in mice treated with ampicillin. Antimicrobial resistance determinants are naturally present in bacterial communities of diverse environments, including the human gut.²⁰ The gut microbiome carries an endogenous pool of ARG that can be rapidly expanded upon exposure to exogenous antimicrobials due to selection and outgrowth of resistant variants.^{5,21} Importantly, it has been shown that exposure to one class of antibiotics can also lead to the enrichment of ARG of unrelated classes of antibiotics and increase the abundance of mobile genetic elements, a phenomenon that is thought to contribute to the emergence of multidrug-resistant pathogens through HGT.^{1,6}

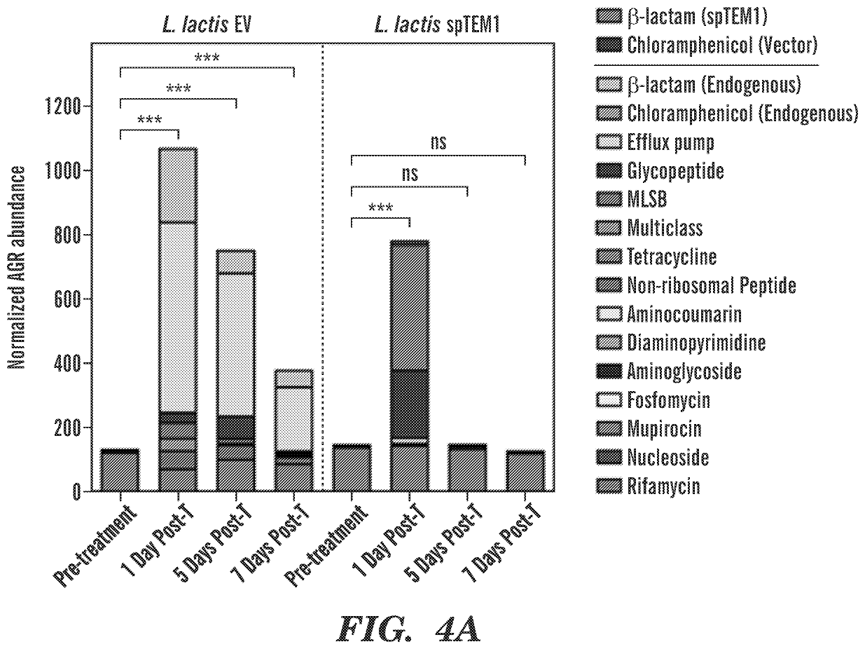

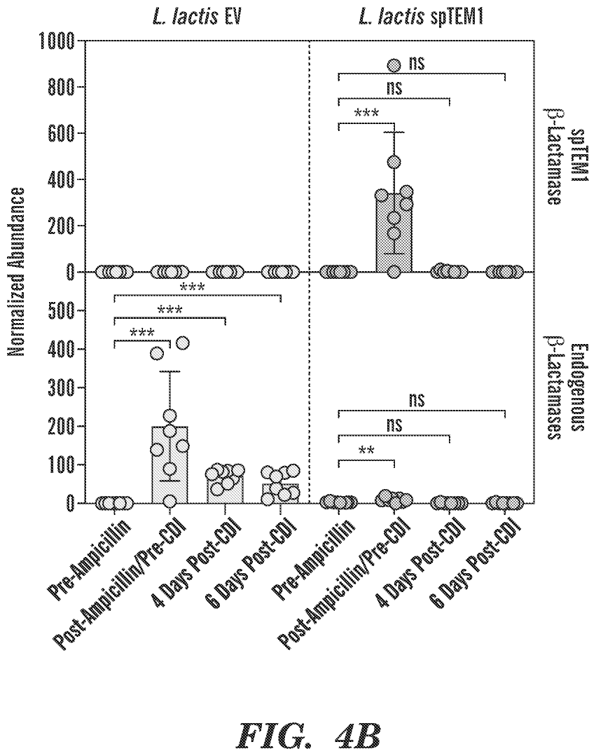

It was investigated whether the expression of the spTEM1 β-lactamase as a public good for the entire gut microbial community removes the selective pressure on ARG upon exposure to ampicillin. For this, metagenomic shotgun sequencing was performed and the reads were mapped to the CARD database²² to determine the abundance of ARG in the mouse fecal samples. It was found that, despite ampicillin treatment, the presence of the L. lactis spTEM1 strain prevented the expansion and subsequent selection of ARG from the endogenous pool of the gut microbiome (FIG. 4). In contrast, the ampicillin treatment in mice that received the L. lactis EV strain resulted in a significant expansion of ARG related not only to β-lactam antibiotics, but also to glycopeptides, non-ribosomal peptides and the macrolide-lincosamide-streptogramin B (MLSB) group as well as broad-substrate efflux pumps (FIG. 4). This analysis detected a number of vector-derived sequences from the spTEM1 system genes and a Chloramphenicol acetyltransferase gene (cat) that mapped to the CARD database. These vector-derived reads were only present in significant numbers at day 1 post-treatment due to lingering L. lactis spTEM1 cells from the last probiotic dose. Importantly, the genes derived from spTEM1 β-lactamase were undetectable by day 5 post treatment, which contrasts with the strong selection and enrichment that was observed for endogenous β-lactamase genes in the L. lactis EV group (FIG. 4). These results indicate that the expression of the spTEM1 system does not confer a selective advantage to producer cells in vivo and demonstrates that β-lactamase activity as a public good removes the selective pressure of ampicillin from the microbial population preventing the enrichment of ARG.

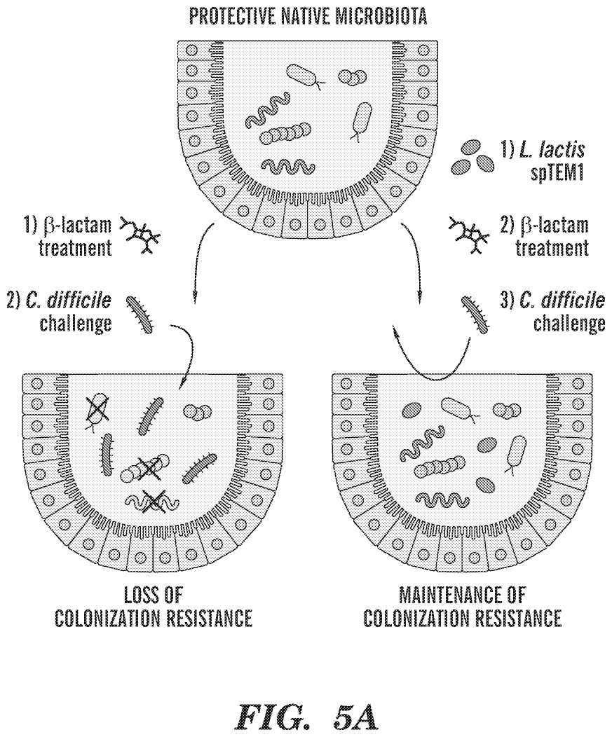

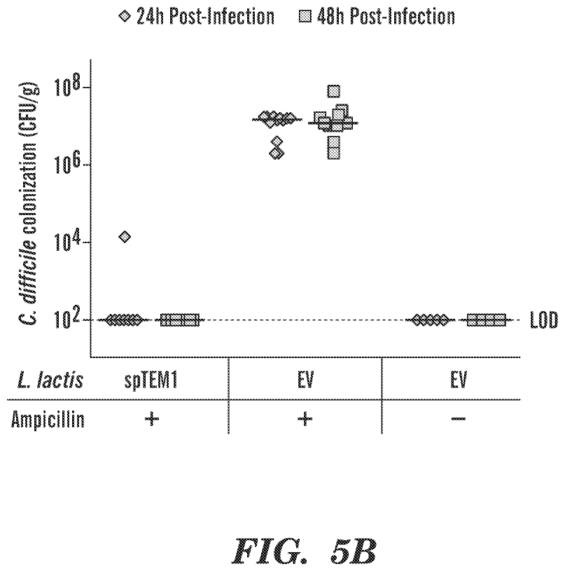

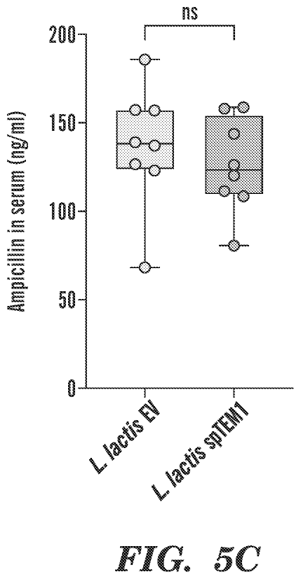

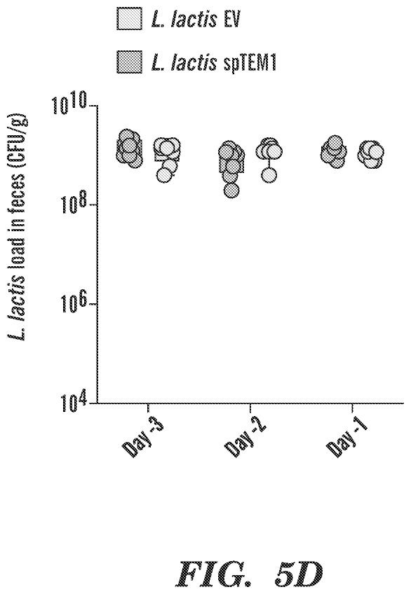

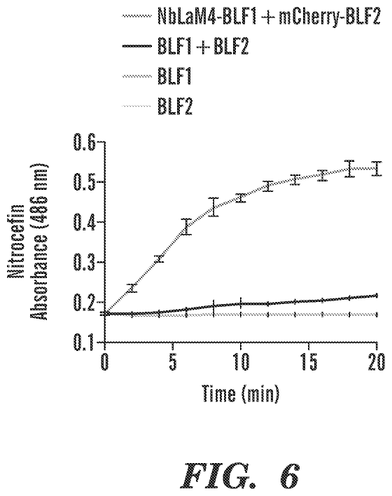

L. lactis spTEM1 maintains the colonization resistance against C. difficile in ampicillin-treated mice. It was evaluated whether the protection of the gut microbiota afforded by the L. lactis spTEM1 strain is enough to maintain colonization resistance against C. difficile after the ampicillin course (FIG. 5A). For this, mice were challenged with 5×10³ spores of C. difficile at day 1 post-treatment and the pathogen colonization at 24- and 48-hours post-infection was evaluated. It was found that the control strain, L. lactis EV, is not capable of maintaining colonization resistance in ampicillin-treated mice nor it confers sensitivity to C. difficile infection in ampicillin-free mice, which indicates that the L. lactis chassis is orthogonal to the infection process (FIG. 5B). Remarkably, the L. lactis spTEM1 strain preserved colonization resistance in all of the ampicillin-treated mice, which demonstrates that the observed level of protection to the diversity and composition of the native microbiota (FIG. 3) is enough to maintain the endogenous factors that determine the exclusion of C. difficile from the gut (FIG. 5B). Importantly, the expression in the gut of extracellular β-lactamase by the L. lactis spTEM1 did not affect the concentration of ampicillin in serum, which ensures that the antibiotic remains effective for its intended purpose of reaching infections outside the gut (FIG. 5C).

Currently there are no FDA-approved drugs to prevent antibiotic-induced dysbiosis. The idea of using purified β-lactamase enzymes to degrade β-lactam antibiotics in the gut was described in 2003.²³ However, only recently a pharmaceutical company has started clinical trials of an encapsulated formulation of the β-lactamase enzyme for direct release in the gut (available on the world wide web at syntheticbiologics.com). The engineered probiotic strategy described herein has two main advantages over the purified enzyme approach. First, the production cost of a probiotic formulation is significantly lower than the one associated to a biologic medicine (i.e. clinical grade enzymes). This factor is especially important for preventative medicines where the cost cannot be great than the β-lactam treatment course or the cost of treatment of potential resultant C. difficile infections. Although cost is an important factor when deciding whether this preventative might be included within the diagnosis related group (DRG) of C. difficile infections by insurance companies, there are benefits of preventing antibiotic-induced dysbiosis beyond the reduction of C. difficile infection alone, because dysbiosis is correlated with numerous other diseases. The low production cost and scalability of probiotic formulations will enable the widespread use of preventatives for antibiotic-induced dysbiosis. Second, this approach is likely to be much more effective than current probiotics, because the use of engineered bacteria as living medicines enables a sustained release of the active enzyme to the intestines as it capitalizes on the ecological properties and metabolic activity of probiotics in the gastrointestinal tract.

It is hypothesized that the use of an engineered probiotic that has a clear mechanism of action, consistent formulation and potency, and defined dosage, the approach described herein will have significantly better performance than currently used probiotics in terms of protection against general dysbiosis and the development of C. difficile. By protecting patients from C. difficile infection, this engineered probiotic has the potential to shorten hospitalization times, reduce morbidity and mortality, and lower the costs associated with the unintentional consequences of IV β-lactam antibiotic use. It is estimated that hospital-acquired C, difficile infections generate ˜$1 billion in attributable added healthcare costs annually. Therefore, a product derived from this engineered probiotic has high clinical relevance, tremendous commercial application, and economic potential.

### Methods

Bacterial strains. Lactococcus lactis NZ3000 (MoBiTec, Germany), a strain for food-grade selection based upon the ability to grow on lactose, was used as the parental strain for recombinant gene expression and animal experiments. L. lactis was maintained at 30° C. in Difco M17 medium supplemented with 0.5% lactose (LM17). Escherichia coli NEB 10-beta (New England BioLabs) cells were used as intermediate hosts for plasmid construction and for assessing survival in cells expressing the spTEM1 system. E. coli cells were propagated at 37° C. in LB broth. Clostridiodes difficile 630 spores were prepared from a single batch and stored long term at 4° C. as previously reported.²⁴

Construction of L. lactis spTEM1. A two-plasmid system was used to achieve expression of the spTEM1 β-lactamase from two independent genetic loci. The ST-BLF-1 fragment was expressed from the plasmid pNZ-ST-BLF-1, which is a derivative of pNZ2122 (MoBiTec, Germany) that uses the pepN promoter instead of the lacA promoter carried in the base plasmid. The SC-BLF-2 fragment was expressed from the plasmid pLL-SC-BLF-2, which is derived from pECGMC²⁵ a shuttle vector with a ColE1 origin of replication for propagation in E. coli, an AMβ1 origin of replication for L. lactis, and a chloramphenicol-resistance gene cat that can be used for selection in both E. coli and L. lactis. L. lactis EV carries pNZ2122 without any insert and served as the empty vector control.

β-lactamase assay. Nitrocefin (BioVision) was dissolved in DMSO to make a 1 mM stock. It was added to samples bacterial culture supernatant in a clear-bottom 96-well plate to a final concentration of 0.1 mM. The plate was placed in a spectrophotometer SpectraMaX™ M5 (Molecular Devices) to read absorbance at 486 nm over time.

Mouse manipulations. 6- to 8-week-old female C57BL/6 mice (Charles River Laboratories) were used for the experiments and had a 5-day acclimation period upon arrival to the mouse facility before the beginning of the experiments. Mice were administered of ampicillin (Patterson Veterinary, USA) via intraperitoneal injection. All mice in this study were treated in accordance with protocol IS00000852-3, approved by Harvard Medical School Institutional Animal Care and Use Committee and the Committee on Microbiological Safety.

Preparation of L. lactis doses. L. lactis cultures were grown to late exponential phase and harvested by centrifugation at 5000×g for 10 minutes at room temperature. Pellets were washed with fresh media and harvested again. Cell pellets were resuspended in a volume that achieved 100-fold concentration using LM17 media supplemented with 200 mM phosphate buffer at pH 7.0 to prevent rapid acidification in the concentrated samples. 1M stock of the phosphate buffer was made with KH₂PO₄ 4.68 g+Na₂HPO₄ 16.4 g in 100 ml DI water (recipe from Sigma Aldrich). The probiotic doses were administered in volumes of 150 μL via oral gavage.

C. difficile infection. A total of 5×10³ spores of C. difficile strain 630 were delivered to mice via oral gavage. Antibiotic-treated mice were given 24 h to recover prior to the infection with C. difficile. To monitor C. difficile colonization, fecal samples were collected, weighed and diluted under anaerobic conditions with anaerobic phosphate-buffered saline. The number of colony forming units (CFU) was counted using TCCFA plates supplemented with 50 μg/mL Erythromycin at 37° C. under anaerobic conditions, as previously reported.²⁶

16S rDNA data processing. 16S data were received as de-multiplexed sequences and were processed using QIIME 2 (Core 2020.2 distribution).²⁷ First, de-multiplexed sequences were stored in a QIIME 2 artifact using the qiime tools import command with parameters—type ‘SampleData[PairedEndSequencesWithQuality]’—input-format PairedEndFastqManifestPhred33V2. In order to remove diversity regions and primers from subsequent analyses, the inventors trimmed the first 23 bp and 20 bp from forward and reverse reads, respectively. Additionally, reads were truncated when the median read quality, from a random subset of 10,000 reads, consistently fell below 30; this quality threshold corresponded to truncating forward reads at 275 bp and reverse reads at 220 bp. Processed sequences (i.e. quality trimmed and filtered) were used to create a feature table containing Amplicon Sequence Variants (ASVs)—equivalent to OTUs clustered at 100% similarity. The aforementioned denoising and feature table construction, in addition to dereplication and chimera removal, were carried out using the QIIME 2 DADA2 plugin.²⁸ The DADA2 plugin pipeline was implemented with the qiime dada2 denoise-paired command with parameters—p-trim-left-f23—p-trim-left-r 20—p-trunc-len-f 275—p-trunc-len-r 220.

Taxonomic classification. In order to assign taxonomies to the processed sequences, a Naive Bayes classifier trained on the Greengenes™ v13.8 99% OTU dataset^{29,30} was used. From the Greengenes™ dataset, the inventors extracted reference reads corresponding to the 16S region amplified in the dataset using the qiimefeature-classifier extract reads command with parameters —p-f-primer GTGCCAGCMGCCGCGGTAA (SEQ ID NO: 9) —p-r-primer GGACTACHVGGGTWTCTAAT (SEQ ID NO: 10). This reference read extraction procedure helps increase the prediction accuracy of the Naïve Bayes classifier by ensuring that the reference reads used for training closely match the 16S region amplified and processed in the dataset. Taxonomic predictions were then carried out with the qiime feature-classifier classify-sklearn command³¹ with default parameters.

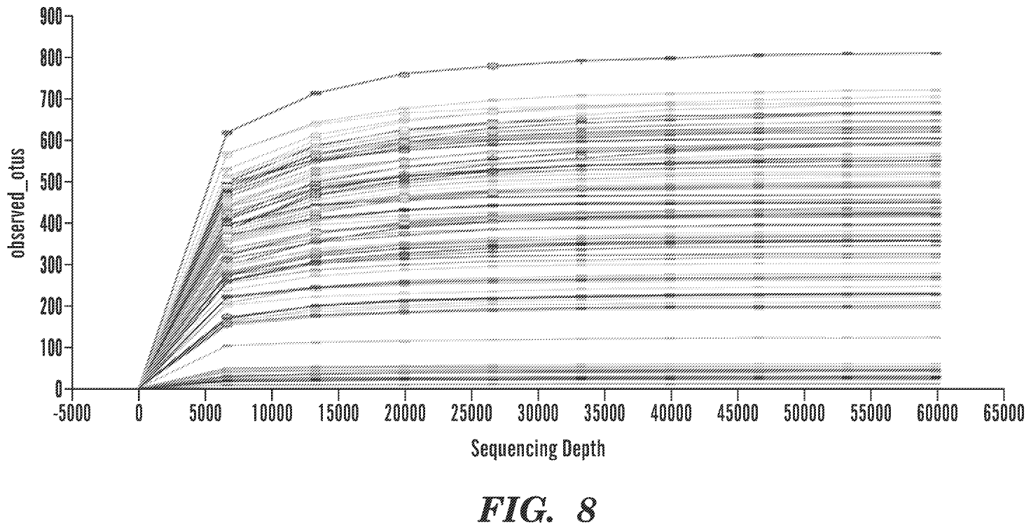

Microbial community analysis. Alpha- and beta-diversity analyses were carried out using the QIIME 2 diversity plugin. First, a tree was created by i) performing a multiple sequence alignment of representative sequences using MAFFT^{32,33} and ii) filtering the alignment to remove highly variable positions which subsequently enabled the construction of an unrooted and rooted tree using the FastTree™ program.^{34,35} This tree-creation pipeline was implemented with the qiime phylogeny align-to-tree-mafft-fasttree command with default parameters. Alpha- and beta-diversity metrics were then calculated using the qiime diversity core-metrics-phylogenetic command with —p-sampling-depth 59900. A sampling depth of 59900 was chosen as it was approximately the largest depth possible without excluding any samples. Rarefaction curves validated that species richness was saturated for all samples well before this sampling depth (FIG. 8). Hence, alpha- and beta-diversity values calculated at this depth are expected to be representative of each samples' microbial communities.

For beta-diversity plots displaying only SpTEM1 and WT treatment groups, only the feature table subset containing data for the SpTEM1 and WT treatment groups was passed through the pipeline outlined above. More specifically, the feature table was filtered for the desired SpTEM1 and WT samples using the qiimefeature-table filter-samples command. Then, alpha- and beta-diversity metrics were calculated using the qiime diversity core-metrics-phylogenetic command with —p-sampling-depth 59900. Distances from baseline values were obtained by calculating the distance in microbial community composition (e.g., Bray-Curtis dissimilarity³⁶) between each sample at day 4, 8, or 10 to each sample's respective microbial community composition at day 0 (e.g., distance between animal number 1 at day 4 to animal number 1 at day 0).

Alpha-diversity values produced by the qiime diversity core-metrics-phylogenetic command were exported to and plotted using PRISM v8.4.1 (GraphPad™; San Diego, CA). Similarly, beta-diversity principal coordinate values were exported to and plotted using GraphPad™ Prism 8 (GraphPad, USA).

Differential abundance. Differential abundance was calculated using analysis of composition of microbiomes (ANCOM)³⁷ which was implemented with the QIIME 2 composition plugin. For pairwise comparisons, the feature table was filtered in order to obtain the treatment groups and/or time-points of interest. All comparisons were carried out at the family level (i.e. level 5 of Greengenes™ taxonomy). In order to conduct comparisons at the family level, feature tables were collapsed at the family level using the qiime taxa collapse command with parameters —p-level 5. Pseudo-counts were added to this collapsed table using the qiime composition add-pseudocount command with default parameters. Lastly, ANCOM was carried out using the qiime composition ancom command with default parameters.

Metagenomic analysis. Reads were first trimmed and filtered using Cutadapt³⁸ v1.18 and Sickle³⁹ v1.33 with parameters pe -q 20-l 30. Host DNA was removed using paired-end mapping with Bowtie2⁴⁰ v2.2.6 against mouse reference genome GCF_000001635.26_GRCm38.p6. In brief, a bowtie sequence index database was created from the mouse reference genome using bowtie2-build with default parameters and paired-end sequences were mapped to this database using bowtie2-x with default parameters. Unmapped reads, in which both reads were unmapped to the mouse reference genome, were extracted using SAMtools⁴¹ v1.6 with parameters view -b -f 12 -F 256.

In order to obtain antibiotic resistance gene (ARG) abundances, filtered reads were mapped to the Comprehensive Antibiotic Resistance Database (CARD)²² protein homolog model version v3.0.9. The CARD protein homolog model contains over 2000 curated sequences of bacterial genes conferring antibiotic resistance. To this CARD protein homolog model sequence database, the inventors manually included three additional CDSs corresponding to probiotic-derived genes coding for chloramphenicol resistance and each half of the split $-lactamase enzyme. Including these three probiotic-derived CDSs helped distinguish ARG reads originating from our engineering probiotics and commensal gut microbiota—the combination of the CARD protein homolog model and the three appended probiotic-derived CDSs is hereafter referred to as the “custom CARD database.” In order to map reads onto this database, a bowtie sequence index database was created from sequences contained in the custom CARD database using bowtie2-build with default parameters. Paired end sequences were mapped to the custom CARD database with Bowtie2 using parameters -D 20 -R 3 -N 1 -L 20 -i S,1,0.50 and SAMtools™ was then used to filter and count the number of reads that mapped to sequences in custom CARD database.

For each sample, mapped read counts (C) for each gene were normalized by gene length (L) and total number of filtered forward and reverse reads (R) using

$${C}_{n o r m} = \frac{C}{( \frac{L}{1 {0}^{3}} ) ( \frac{R}{1 {0}^{6}} )}$$

where Cₙₒᵣₘ represents normalized gene count. Gene length and number of reads were divided by 10³ and 10⁶, respectively, in order to represent gene length in kilobases and number of reads in millions.

Genes with normalized counts >0 in any sample were kept for further analyses. 219 genes in the custom CARD database satisfied this criterion and one sample (T4-9) was removed as it contained no sequencing reads. Using annotations available in CARD, these 219 genes were manually grouped into 15 major resistance classes: aminocoumarin, aminoglycoside, β-lactam, chloramphenicol, diaminopyrimidine, efflux pump, fosfomycin, glycopeptide, mlsb (macrolide, lincosamide, or streptogramin B), multiclass (i.e. resistance to multiple antibiotic classes, excluding efflux pumps), mupirocin, non-ribosomal peptide, nucleoside, rifamycin, and tetracycline. For each treatment group and time point, stacked bar plots were created by summing normalized gene counts for each major resistance class within and across samples, and then dividing by the number of samples in each respective treatment group. Statistical analysis was done in R⁴² v.3.6.1 and plots were generated using GraphPad™ Prism 8 (GraphPad, USA). Statistical significance of differences in normalized abundance for each resistance class was assessed using negative binomial generalized linear models in the MASS package⁴³ v7.3-51.6 with Tukey's post-hoc test.⁴⁴ ARG annotation tables were organized into data objects using phyloseq.⁴⁵

In order to calculate abundance of engineered probiotic-derived plasmid in samples and identify potential horizontal gene transfer (HGT) events, the inventors conducted a BLAST™ (Basic Local Alignment Search Tool)⁴⁶ search against plasmid sequences using BLAST+™ 47 v2.7.1. In this BLAST™ search, sample reads were used as query sequences against a database consisting of the four plasmids used for engineered probiotic and wild type control strains. The BLAST™ database consisting of plasmid sequences used in probiotic strains was created using the makeblastdb. Since the plasmids used in each treatment group shared a backbone region, a “hit” was counted only when the corresponding query read mapped to a plasmid from the sample's treatment group. For each sample, hits were normalized by dividing by the total number of forward and reverse reads.

### REFERENCES

1. Becattini, S., Taur, Y. & Pamer, E. G. Antibiotic-Induced Changes in the Intestinal Microbiota and Disease. Trends Mol Med 22, 458-478 (2016).
2. Karachalios, G. & Charalabopoulos, K. Biliary Excretion of Antimicrobial Drugs. Chemotherapy 48, 280-297 (2002).
3. Ghibellini, G., Leslie, E. M. & Brouwer, K. L. Methods to evaluate biliary excretion of drugs in humans: an updated review. Mol Pharm 3, 198-211 (2006).
4. Stecher, B., Maier, L. & Hardt, W. D. ‘Blooming’ in the gut: how dysbiosis might contribute to pathogen evolution. Nat Rev Microbiol 11, 277-284 (2013).
5. Modi, S. R., Lee, H. H., Spina, C. S. & Collins, J. J. Antibiotic treatment expands the resistance reservoir and ecological network of the phage metagenome. Nature 499, 219-222 (2013).
6. Kent, AG., Vill, A. C., Shi, Q., Satlin, M. J. & Brito, I. L. Widespread transfer of mobile antibiotic resistance genes within individual gut microbiomes revealed through bacterial Hi-C. Nat Commun 11, 4379 (2020).
7. WHO. WHO report on surveillance of antibiotic consumption: 2016-2018 early implementation. Geneva: World Health Organization; 2018. Licence: CC BY—NC-SA 3.0 IGO. (2018).
8. Goldenberg, J. Z., et al. Probiotics for the prevention of Clostridium difficile-associated diarrhea in adults and children. Cochrane Database Syst Rev 12, CD006095 (2017).
9. Fisher, J. F. & Mobashery, S. beta-Lactam Resistance Mechanisms: Gram-Positive Bacteria and Mycobacterium tuberculosis. Cold Spring Harb Perspect Med 6(2016).
10. Wright, G. D. Bacterial resistance to antibiotics: enzymatic degradation and modification. Adv Drug Deliv Rev 57, 1451-1470 (2005).
11. Bush, K. Past and Present Perspectives on beta-Lactamases. Antimicrob Agents Chemother 62(2018).
12. Bush, K. & Bradford, P. A. Epidemiology of beta-Lactamase-Producing Pathogens. Clin Microbiol Rev 33(2020).
13. Teuber, M. The genus Lactococcus. in The Genera of Lactic Acid Bacteria (eds. Wood, B. J. B. & Holzapfel, W. H.) 173-234 (Springer US, Boston, MA, 1995).
14. Zhang, C., et al. Ecological robustness of the gut microbiota in response to ingestion of transient food-borne microbes. ISMEJ 10, 2235-2245 (2016).
15. Limaye, S. A., et al. Phase 1b, multicenter, single blinded, placebo-controlled, sequential dose escalation study to assess the safety and tolerability of topically applied AGO13 in subjects with locally advanced head and neck cancer receiving induction chemotherapy. Cancer 119, 4268-4276 (2013).
16. Braat, H., et al. A phase I trial with transgenic bacteria expressing interleukin-10 in Crohn's disease. Clin Gastroenterol Hepatol 4, 754-759 (2006).
17. Galarneau, A., Primeau, M., Trudeau, L.-E. & Michnick, S. W. β-Lactamase protein fragment complementation assays as in vivo and in vitro sensors of protein-protein interactions. Nature Biotechnology 20, 619-622 (2002).
18. Zakeri, B., et al. Peptide tag forming a rapid covalent bond to a protein, through engineering a bacterial adhesin. Proc Natl Acad Sci USA 109, E690-697 (2012).
19. Nielsen, J. B. & Lampen, J. O. Membrane-bound penicillinases in Gram-positive bacteria. J Biol Chem 257, 4490-4495 (1982).
20. Forsberg, K. J., et al. The shared antibiotic resistome of soil bacteria and human pathogens. Science 337, 1107-1111 (2012).
21. Jernberg, C., Lofmark, S., Edlund, C. & Jansson, J. K. Long-term ecological impacts of antibiotic administration on the human intestinal microbiota. ISMEJ 1, 56-66 (2007).
22. Alcock, B. P., et al. CARD 2020: antibiotic resistome surveillance with the comprehensive antibiotic resistance database. Nucleic Acids Res 48, D517-D525 (2020).
23. Harmoinen, J., et al. Enzymic degradation of a beta-lactam antibiotic, ampicillin, in the gut: a novel treatment modality. J Antimicrob Chemother 51, 361-365 (2003).
24. Edwards, A. N. & McBride, S. M. Isolating and Purifying Clostridium difficile Spores. Methods Mol Biol 1476, 117-128 (2016).
25. Cameron, D. E. & Collins, J. J. Tunable protein degradation in bacteria. Nat Biotechnol 32, 1276-1281 (2014).
26. Winston, J. A., Thanissery, R., Montgomery, S. A. & Theriot, C. M. Cefoperazone-treated Mouse Model of Clinically-relevant Clostridium difficile Strain R20291. J Vis Exp (2016).
27. Bolyen, E., et al. Reproducible, interactive, scalable and extensible microbiome data science using QIIME 2. Nature Biotechnology 37, 852-857 (2019).
28. Callahan, B. J., et al. DADA2: High-resolution sample inference from Illumina amplicon data. Nature Methods 13, 581-583 (2016).
29. DeSantis, T. Z., et al. Greengenes, a Chimera-Checked 16S rRNA Gene Database and Workbench Compatible with ARB. Appl. Environ. Microbiol. 72, 5069-5072 (2006).
30. McDonald, D., et al. An improved Greengenes taxonomy with explicit ranks for ecological and evolutionary analyses of bacteria and archaea. The ISME Journal6, 610-618 (2012).
31. Bokulich, N. A., et al. Optimizing taxonomic classification of marker-gene amplicon sequences with QIIME 2's q2-feature-classifier plugin. Microbiome 6, 90 (2018).
32. Kuraku, S., Zmasek, C. M., Nishimura, 0. & Katoh, K. aLeaves facilitates on-demand exploration of metazoan gene family trees on MAFFT sequence alignment server with enhanced interactivity. Nucleic Acids Res. 41, W22-28 (2013).
33. Katoh, K., Rozewicki, J. & Yamada, K. D. MAFFT online service: multiple sequence alignment, interactive sequence choice and visualization. Brief Bioinformatics 20, 1160-1166 (2019).
34. Price, M. N., Dehal, P. S. & Arkin, A. P. FastTree: computing large minimum evolution trees with profiles instead of a distance matrix. Mol. Biol. Evol. 26, 1641-1650 (2009).
35. Price, M. N., Dehal, P. S. & Arkin, A. P. FastTree 2 —Approximately Maximum-Likelihood Trees for Large Alignments. PLOS ONE 5, e9490 (2010).
36. Bray, J. R. & Curtis, J. T. An Ordination of the Upland Forest Communities of Southern Wisconsin. Ecological Monographs 27, 325-349 (1957).
37. Mandal, S., et al. Analysis of composition of microbiomes: a novel method for studying microbial composition. Microbial Ecology in Health and Disease 26, 27663 (2015).
38. Martin, M. Cutadapt removes adapter sequences from high-throughput sequencing reads. EMBnet.journal 17, 10-12 (2011).
39. Joshi, N. A. & Fass, J. N. Sickle: A sliding-window, adaptive, quality-based trimming tool for FastQ files.
40. Langmead, B. & Salzberg, S. L. Fast gapped-read alignment with Bowtie 2. Nature Methods 9, 357-359 (2012).
41. Li, H., et al. The Sequence Alignment/Map format and SAMtools. Bioinformatics 25, 2078-2079 (2009).
42. Team, R. C. R: A Language and Environment for Statistical Computing. (R Foundation for Statistical Computing).
43. Venables, W. N. & Ripley, B. D. Modern Applied Statistics with S-PLUS, (Springer Science & Business Media, 2013).
44. Miller, R. G. Introduction. in Simultaneous Statistical Inference (ed. Miller, R. G.) 1-35 (Springer, NewYork, NY, 1981).
45. McMurdie, P. J. & Holmes, S. phyloseq: An R Package for Reproducible Interactive Analysis and Graphics of Microbiome Census Data. PLOS ONE 8, e61217 (2013).
46. Altschul, S. F., Gish, W., Miller, W., Myers, E. W. & Lipman, D.J. Basic local alignment search tool. J. Mol. Biol. 215, 403-410 (1990).
47. Camacho, C., et al. BLAST+: architecture and applications. BMC Bioinformatics 10, 421 (2009).

### Example 2: Split Inteins

The inventors generated a C-terminal fusion of the N-terminus of the TEM1 enzyme with the CfaN fragment of a split intein. Likewise, an N-terminal fusion containing the CfaC fragment of the reported split intein and the C-terminus of the TEM1 was created. Nitrocefin hydrolysis assays showed low levels of the reconstituted spTEM1 demonstrating feasibility of the approach.

1. A composition comprising a microorganism engineered to degrade an antibiotic in the mammalian gut, wherein the microorganism is also engineered to reduce the likelihood of horizontal transmission of its engineered antibiotic-degrading capacity.

2. The composition of claim 1, wherein the microorganism's engineered antibiotic degrading capacity comprises expression and secretion of an enzyme activity that degrades the antibiotic.

3. The composition of claim 2, wherein the enzyme is encoded in first and second parts, on separate, first and second nucleic acid constructs, wherein neither construct on its own encodes active antibiotic-degrading enzyme, and where both parts of the enzyme are needed to provide antibiotic-degrading activity, thereby reducing the likelihood of horizontal transmission of the engineered antibiotic-degrading activity.

4. The composition of any one of claims 1-3, wherein the microorganism is a bacterium or a yeast.

5. The composition of claim 3 or claim 4, wherein the first and second constructs encode the first and second parts of the enzyme as first and second fusion polypeptides, each comprising a respective member of a specific binding pair.

6. The composition of claim 5, wherein the first and second fusion polypeptides are secreted by the microorganism into its surrounding environment.

7. The composition of claim 5 or claim 6, wherein binding of the first and second fusion polypeptides via the respective members of the specific binding pair promotes the physical interaction of the first and second parts of the enzyme and reconstitution of antibiotic-degrading enzymatic activity.

8. The composition of any one of claims 5-7, wherein the respective members of the specific binding pair promote covalent bonding between the first and second fusion polypeptides.

9. The composition of any one of claims 1-8, wherein the antibiotic-degrading activity comprises β-lactamase enzyme activity, Staphylococcus aureus mph(C) gene enzyme activity, S. aureus lincosamide nucleotidyltransferase lnu(A) gene enzyme activity, an Enterococcus faecium lnu(b) gene enzyme activity or an Escherichia coli ereB gene enzyme activity.

10. The composition of claim 9, wherein the β lactamase is a TEM1 β lactamase.

11. The composition of claim 10, wherein the TEM1 β lactamase is encoded in first and second parts, on separate, first and second nucleic acid constructs, wherein the first nucleic acid construct encodes β lactamase fragment (BLF) 1, comprising SEQ ID NO: 1 or 3, and the second nucleic acid construct encodes BLF 2, comprising SEQ ID NO: 5 or 7.

12. The composition of any one of claims 3-11, wherein the first and second constructs encode the first and second parts of the enzyme as first and second fusion polypeptides, respectively, each comprising a respective member of the SpyTag/SpyCatcher specific binding pair.

13. The composition of any one of claims 1-12, wherein the microorganism is an engineered generally regarded as safe (GRAS) microorganism.

14. The composition of any one of claims 1-13, wherein the microorganism is an engineered lactic acid bacterium.

15. The composition of any one of claims 1-14, wherein the microorganism is an engineered Lactococcus lactis bacterium.

16. The composition of any one of claims 1-15, in a formulation for oral delivery.

17. A viable lyophilized microorganism as recited in of any one of claims 1-16.

18. The composition of any one of claims 1-17, formulated as a pill, tablet or capsule.

19. A method of treating a bacterial infection, the method comprising administering an antibiotic and a composition of any one of claims 1-18.

20. The method of claim 19, wherein the antibiotic is delivered parenterally or orally.

21. The method of claim 19 or claim 20, wherein the antibiotic is delivered intravenously.

22. The method of any one of claims 19-21, wherein the composition is administered before the antibiotic is administered.

23. The method of any one of claims 19-22, wherein the antibiotic is delivered parenterally, and the composition is administered before or at the same time the antibiotic is delivered.

24. The method of any one of claims 19-23, wherein the composition is administered orally.

25. The method of claim 19 or claim 20, wherein the composition is orally administered before the antibiotic is orally administered.

26. The method of any one of claims 19-25 wherein the antibiotic is a β-lactam antibiotic, and the microorganism in the composition is engineered to express a β-lactamase enzyme.

27. The method of any one of claims 19-26, wherein the antibiotic is selected from penicillin, penicillin G, penicillin V, oxacillin, nafcillin, dicloxacillin, amoxicillin, ampicillin, ticarcillin, piperacillin, cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, ceftobiprole, meropenem, doripenem, ertapenam, or aztreonam).

28. The method of any one of claims 19-27, wherein the microorganism in the composition promotes the degradation of the antibiotic in the gut, thereby limiting or preventing antibiotic-induced gut dysbiosis.

29. A method of preventing or limiting an antibiotic-induced dysbiosis in a subject in need of antibiotic administration, the method comprising administering an antibiotic and a composition of any one of claims 1-18.

30. The method of claim 29, wherein the composition is administered orally.

31. The method of claim 29 or 30, wherein the antibiotic is administered parenterally or orally.

32. The method of any one of claims 29-31, wherein the composition is orally administered before the antibiotic is orally administered.

33. The method of any one of claims 29-32, wherein the bacterial infection is an infection with a bacterium sensitive to a β-lactam antibiotic, and the antibiotic is a β-lactam antibiotic.

34. The method of any one of claims 29-33, wherein the antibiotic is selected from penicillin, penicillin G, penicillin V, oxacillin, nafcillin, dicloxacillin, amoxicillin, ampicillin, ticarcillin, piperacillin, cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, ceftobiprole, meropenem, doripenem, ertapenam, or aztreonam).

35. A method of preventing C. difficile pathology in a subject treated with an antibiotic, the method comprising administering a composition of any one of claims 1-18 to the subject.

36. The method of claim 35, wherein the composition is administered orally.

37. The method of claim 35 or 36, wherein the antibiotic is administered parenterally or orally.

38. The method of any one of claims 35-37, wherein the composition is orally administered before the antibiotic is orally administered.

39. The method of any one of claims 35-38, wherein the subject comprises a bacterial infection comprising a bacterium sensitive to a β-lactam antibiotic, and the antibiotic is a β-lactam antibiotic.

40. The method of any one of claims 35-39, wherein the antibiotic is selected from penicillin, penicillin G, penicillin V, oxacillin, nafcillin, dicloxacillin, amoxicillin, ampicillin, ticarcillin, piperacillin, cefazolin, cephalexin, cefadroxil, cefuroxime, cefoxitin, cefotetan, cefaclor, cefprozil, cefotaxime, ceftriaxone, cefpodoxime, cefixime, cefdinir, cefditoren, ceftibuten, ceftazidime, cefepime, ceftaroline, cefiderocol, ceftobiprole, meropenem, doripenem, ertapenam, or aztreonam.

41. A system for limiting or preventing antibiotic-induced dysbiosis, the system comprising
a first nucleic acid construct, encoding a first fusion polypeptide comprising a first part of an antibiotic-degrading enzyme, fused to a first member of a specific binding pair, operably linked to sequence permitting expression of the first fusion polypeptide;
a second nucleic acid construct, encoding a second fusion polypeptide comprising a second part of an antibiotic-degrading enzyme, fused to the second member of the specific binding pair, operably linked to sequence permitting expression of the second fusion polypeptide,
wherein neither the first fusion polypeptide nor the second fusion polypeptide alone can degrade antibiotic, but wherein a physical association between the first and second fusion polypeptides permits association between the first and second parts of the antibiotic-degrading enzyme to form an active antibiotic-degrading complex.

42. The system of claim 41, comprised by a microorganism.

43. The system of claim 41, wherein the microorganism is a bacterium or a yeast.

44. The system of any one of claims 41-43, wherein the respective members of the specific binding pair promote covalent bonding between the first and second fusion polypeptides.

45. The system of any one of claims 41-44, wherein the antibiotic-degrading activity comprises a β lactamase enzyme activity, Staphylococcus aureus mph(C) gene enzyme activity, S. aureus lincosamide nucleotidyltransferase lnu(A) gene enzyme activity, an Enterococcus faecium lnu(b) gene enzyme activity or an Escherichia coli ereB gene enzyme activity.

46. The system of claim 45, wherein the β lactamase is a TEM1 β lactamase.

47. The system of claim 41, wherein the first nucleic acid construct comprises sequence encoding β lactamase fragment (BLF) 1, comprising SEQ ID NO: 1 or 3, and the second nucleic acid construct comprises sequence encoding BLF 2, comprising SEQ ID NO: 5 or 7.

48. The system of any one of claims 41-47, wherein the specific binding pair is the respective members of the SpyTag/SpyCatcher specific binding pair.

49. The system of any one of claims 41-48, wherein the microorganism is an engineered GRAS microorganism.

50. The system of any one of claims 41-49, wherein the microorganism is a lactic acid bacterium.

51. The system of any one of claims 41-50, wherein the microorganism is a Lactococcus lactis bacterium.
